(19) United States
(12) Patent Application Publication
(10) Pub. No.: US 2012/0164128 A1
(43) Pub. Date: Jun. 28, 2012

(54) MEANS AND METHODS FOR COUNTERACTING POLYQ EXPANSION DISORDERS

(75) Inventor: Eric Albert Julius Reits, Amsterdam (NL)

(73) Assignee: Academisch Medisch Centrum, Amsterdam Zuidoost (NL)

(21) Appl. No.: 13/321,539

(22) PCT Filed: May 20, 2010
(86) PCT No.: PCT/NL2010/050306

§ 371 Date: Mar. 1, 2012

Related U.S. Application Data
(60) Provisional application No. 61/179,969, filed on May 20, 2009.

(30) Foreign Application Priority Data
May 20, 2009 (EP) 09160830.7

(51) Int. Cl. A61K 38/48; C07K 14/00; G01N 33/53; A61P 25/14; A61P 25/00; A61K 31/711; C07H 21/04

(52) U.S. Cl. 424/94.64; 424/94.63; 514/44 R; 530/350; 536/23.4; 435/7.21

(57) ABSTRACT

The present invention provides means and methods for counteracting and/or preventing aggregation of a polyQ protein. Further provided are improved poly constructs which are, amongst other things, useful for testing assays. According to the invention, several peptidases like, e.g. tripeptidyl peptidase II (TPPII), appear to be capable of cleaving long polyQ peptides comprising at least 45 glutamine residues. Hence, according to the invention, administration of such peptidases to an individual suffering from a polyQ expansion disorder results in degradation of long polyQ peptides.

The invention relates to the fields of biology and medicine.

Numerous neurodegenerative diseases are manifested by the accumulation and aggregation of intracellular proteins. These diseases include polyglutamine (polyQ) expansion disorders like Huntington's disease (HD), Spinal Bulbar Muscular Atrophy (SBMA) and various spinocerebellar ataxia's (SCAs). PolyQ disorders are dominantly inherited and caused by expansions of glutamine-encoding CAG repeats. Normally, the disease-related proteins involved contain sequences of 6-40 glutamine repeats, while expansion of these tracts to 40-300 repeats leads to disease. The age of onset of the disorder is inversely correlated with the repeat length of the polyQ tracts. Clinical features and patterns of neuronal degeneration differ among the various polyglutamine-mediated neurodegenerative disorders but important pathogenic characteristics are common: all are progressive disorders characterized by neuronal dysfunction and neuronal loss progress over 10-30 years after onset.

The expansion of a polyglutamine-encoding CAG repeat results in the extension of a stretch of glutamines in the encoded proteins. Proteins with such enlarged stretch of polyglutamines tend to aggregate and neuronal intracellular inclusions comprising such aggregates are found in diseased individuals, resulting in dysfunctionality and degeneration of the affected neurons.

Currently, polyQ expansion disorders cannot be cured. Treatments, such as for instance diets, tranquillizers, antipsychotics and speech therapy are intended to alleviate the symptoms. Research was, until the present invention, mainly focussed on proteolytic protein fragments harbouring a polyQ tract in aggregates. The common view in the field is that polyQ proteins, and in particular proteolytic fragments thereof, initiate protein aggregation and induce neuronal toxicity. Such protein aggregates were believed to block the ubiquitin-proteasome system (UPS), leading to cell stress and toxicity. While full length polyQ proteins hardly aggregate, aggregation was, before the present invention, thought to be initiated by their proteolytic fragments containing the polyQ tract. These proteolytic fragments can be generated by proteases like caspases, aspartic endopeptidases, calpains and the proteasome. Accumulation of these proteolytic fragments was commonly thought to function as a nucleation centre that sequesters full-length polyQ proteins in time. It was therefore commonly believed that proteases should be inhibited in order to counteract the formation of proteolytic fragments of polyQ proteins, thereby delaying aggregation and disease progression.

Another view in the field is that molecular chaperones are useful in combating polyQ expansion disorders. For instance, international patent application WO 2008/127100 discloses the use of specific members of the Hsp40 heat shock protein family, and use of compounds capable of increasing the amount or activity of such Hsp40 members, for counteracting protein aggregation.

It is an object of the present invention to provide alternative means and methods for counteracting protein aggregation and for counteracting polyQ expansion disorders. It is a further object to provide an improved polyQ construct which is amongst other things useful for testing assays.

The present invention provides a novel approach for counteracting polyQ protein aggregation. The present inventors have focussed on the insight that the proteasome is able to digest polyQ proteins, but not the actual polyQ stretch itself, which is released from the proteasome as a peptide. PolyQ proteins which are not related to disease, containing a polyQ stretch of at most 40 glutamine residues, result in rather short polyQ peptides which are released from the proteasome and degraded by cellular proteases such as the puromycin-sensitive aminopeptidase (PSA) (Bhutani et al, 2007). However, PSA is not capable of degrading long polyQ stretches of disease- related polyQ proteins as it can only digest peptides up to around 40 amino acids. Proteasomal degradation of disease-related polyQ proteins, which contain longer polyglutamine stretches of about 40-300 repeats, results in long polyQ peptides containing about 40-300 glutamine residues. Such long polyQ peptides containing a polyQ stretch of about 40-300 glutamine residues which are released from the proteasome after digestion of disease-related polyQ proteins are not easily degraded and become major aggregation centres which initiate protein aggregation. Therefore, these long polyQ peptides are targeted by the present invention. Methods are provided wherein these long polyQ peptides are degraded or inactivated. Preferably, said long polyQ peptides are degraded. The present invention provides the insight that, even though such long polyQ peptides are not sufficiently removed in cells of individuals suffering from polyQ expansion disorders, they can nevertheless be degraded by several peptidases. According to the invention, several peptidases appear to be capable of cleaving long polyQ peptides comprising at least 45 glutamine residues. Hence, according to the invention, administration of such peptidases to an individual suffering from a polyQ expansion disorder results in degradation of long polyQ peptides. Since such long polyQ peptides containing a polyQ stretch of about 40-300 glutamine residues are major aggregation centres which initiate protein aggregation, degradation of long polyQ peptides diminishes aggregation. The invention therefore provides a use of a peptidase capable of cleaving a polyQ peptide comprising at least 45 glutamine residues for the preparation of a medicament for at least in part treating and/or preventing a disorder associated with polyglutamine-mediated protein aggregation. It is, of course, also possible to use a truncated form of such peptidase, as long as the functional catalytic domain which is capable of cleaving said polyQ peptide is still present. Alternatively, or additionally, a nucleic acid sequence encoding said peptidase or encoding at least said functional catalytic domain is used. After administration of such nucleic acid to an individual, it will be translated by the individual's nucleic acid translation machinery resulting in production of the encoded peptidase or catalytic domain in vivo. Once produced, the peptidase or catalytic domain will degrade long polyQ peptides released from the proteasomes.

In yet another embodiment, a compound that is capable of enhancing the expression, amount and/or peptidase activity of said peptidase is used. In this embodiment, an endogenously present peptidase is upregulated or activated in order to increase the overall peptidase activity. A non-limiting example of a compound capable of enhancing the expression of a peptidase is an enhancer, capable of binding the promoter region of the gene encoding said peptidase, thereby increasing expression. Another non-limiting example of a compound capable of enhancing the expression of a peptidase is a compound which is capable of binding a silencer motif of the gene encoding said peptidase. This way, inhibition of expression is counteracted, resulting in increased expression. A non-limiting example of a compound capable of enhancing the amount of a peptidase is a compound which is capable of increasing the half-life of said peptidase. A non-limiting example of a compound capable of enhancing the peptidase activity of a peptidase is a compound which is capable of altering the conformation or complex formation of said peptidase. Another non- limiting example is a compound capable of counteracting an inhibitor of said peptidase.

The invention thus provides a use of a peptidase capable of cleaving a polyQ peptide comprising at least 45 glutamine residues, or a functional catalytic domain of said peptidase, or a nucleic acid sequence encoding said peptidase or functional catalytic domain, or a compound that is capable of enhancing the expression, amount and/or peptidase activity of said peptidase, for the preparation of a medicament for at least in part treating and/or preventing a disorder associated with polyglutamine- mediated protein aggregation. Also provided is a peptidase capable of cleaving a polyQ peptide comprising at least 45 glutamine residues, or a functional catalytic domain of said peptidase, or a nucleic acid sequence encoding said peptidase or functional catalytic domain, or a compound that is capable of enhancing the expression, amount and/or peptidase activity of said peptidase, for use in at least in part treating and/or preventing a disorder associated with polyglutamine-mediated protein aggregation. A use of said peptidase, catalytic domain, nucleic acid or compound for counteracting and/or preventing a polyglutamine-mediated protein aggregation disorder is also provided. One embodiment thus provides a method for counteracting and/or preventing a polyglutamine-mediated protein aggregation disorder, comprising administering to an individual in need thereof a pharmaceutically effective amount of a peptidase capable of cleaving a polyQ peptide comprising at least 45 glutamine residues, or a functional catalytic domain of said peptidase, or a nucleic acid sequence encoding said peptidase or functional catalytic domain, or a compound that is capable of enhancing the expression and/or peptidase activity of said peptidase. Preferably, said method is performed after said individual has been diagnosed with said disorder associated with polyglutamine-mediated protein aggregation or after said individual has been diagnosed with a risk of developing said disorder. This is for instance done by assessing whether said individual comprises (nucleic acid encoding) a disease- specific poly Q protein containing a polyQ stretch of about 40-300 glutamine residues.

International patent application WO 2009/12176 is concerned with brain diseases, including lysosomal storage diseases such as infantile or late infantile ceroid lipofuscinoses, neuronopathic Gaucher, Juvenile Batten, Fabry, MLD, Sanfilippo, Hunter, Krabbe, Morquio, Pompe and Niemann-Pick. Neurodegenerative diseases including, amongst other things, polyglutamine repeat disease, such as Huntington's disease and Kennedy disease, are also mentioned. WO 2009/12176, however, is directed towards peptides that selectively target therapeutic agents to vascular endothelial cells of the central nervous system. The teaching of W02009/12176 is exemplified by the use of the enzyme tripeptidyl peptidase I for the treatment of late infantile neuronal ceroid lipofuscinosis. However, tripeptidyl peptidase I, which is an extracellular enzyme, is not a peptidase capable of cleaving (intracellular) poly-Q peptides comprising at least 45 glutamine residues. Moreover, late infantile neuronal ceroid lipofuscinosis is not a polyglutamine-mediated protein aggregation disorder. The present invention is thus clearly novel over this teaching. International application WO 2002/055684 is also concerned with brain diseases, such as lysosomal storage diseases and polyglutamine repeat disorders. Gene therapy is disclosed, wherein polynucleotides are used that encode a lysozomal enzyme or a secreted protein such as beta-glucuronidase, pepstatin insensitive protease or palmitoyl protein thioesterase. Of note, WO 2002/055684 does not mention any enzyme that is capable of cleaving a poly-Q peptide comprising at least 45 glutamine residues. On the contrary, upregulation of the lysosomal enzyme beta-glucuronidase is associated with neuronal damage in neurodegenerative disorders such as Alzheimers and Huntingtons Disease. In addition, these secreted or lysosomal enzymes are not able to access and degrade cytoplasmic or nuclear poly-Q peptides.

As used herein, a peptidase capable of cleaving a polyQ peptide comprising at least 45 glutamine residues is defined as an enzyme capable of cleaving at least one bond within said polyQ peptide. Said peptidase is capable of splitting said peptide into at least two smaller parts. Although further degradation of the resulting parts may occur, said peptide need not be capable of totally degrading said polyQ peptide.

A long polyQ peptide is defined as a peptide containing a polyQ stretch with a length of at least 40 glutamine residues. Preferably, said long polyQ peptide comprises a stretch of about 45-300 glutamine residues.

A compound is defined herein as a natural or non-natural molecule or a combination of molecules. A compound for instance comprises a small molecule compound, a peptide, a protein or a nucleic acid molecule, including but not limited to an expression vector, or any combination thereof.

A polyglutamine-mediated disorder is defined as a disorder that is characterized by accumulation of polyglutamine-containing protein. Said polyglutamine-mediated disorder is preferably selected from Huntington's disease, Dentatorubral-pallidoluysian atrophy (DRPLA), X-linked spinal and bulbar muscular atrophy (SBMA) and/or spinocerebellar ataxias (SCA).

As used herein, a nucleic acid molecule or nucleic acid sequence of the invention preferably comprises a chain of nucleotides, more preferably DNA and/or RNA. In other embodiments a nucleic acid molecule or nucleic acid sequence of the invention comprises other kinds of nucleic acid structures such as for instance a DNA/RNA helix, peptide nucleic acid (PNA), locked nucleic acid (LNA) and/or a ribozyme. Hence, the term “nucleic acid molecule” or “nucleic acid sequence” also encompasses a chain comprising non-natural nucleotides, modified nucleotides and/or non-nucleotide building blocks which exhibit the same function as natural nucleotides.

Now that the invention has provided the insight that polyglutamine-mediated disorders are counteracted by using peptidases capable of cleaving polyQ peptides with a length of at least 45 glutamine residues, it has become possible to use different peptidases with this capacity. Preferably, a cysteine peptidase or a serine peptidase is used. In a particularly preferred embodiment, said peptidase capable of cleaving polyQ peptides with a length of at least 45 glutamine residues comprises tripeptidyl peptidase II (TPPII). TPPII is a high molecular weight peptidase which is expressed in a wide range of eukaryotic organisms. TPPII is an aminopeptidase which is capable of cleaving large peptides. It has exopeptidase activity as well as endopeptidase activity and is believed to play a role in the turnover of intracellular proteins. A review of the structure and function of TPPII is provided in Tomkinson et al, 2005. Before the present invention, the capability of TPPII of cleaving long polyQ peptides containing at least 45 glutamine residues was unknown. Further provided is therefore a use of TPPII, or use of a functional catalytic domain of TPPII, or use of a nucleic acid sequence encoding TPPII or a functional catalytic domain of TPPII, or use of a compound that is capable of enhancing the expression, amount and/or peptidase activity of TPPII, for the preparation of a medicament for at least in part treating and/or preventing a disorder associated with polyglutamine-mediated protein aggregation. Also provided is TPPII, or a functional catalytic domain of TPPII, or a nucleic acid sequence encoding TPPII or a functional catalytic domain of TPPII, or a compound that is capable of enhancing the expression, amount and/or peptidase activity of TPPII, for use in at least in part treating and/or preventing a disorder associated with polyglutamine-mediated protein aggregation. A use of said TPPII, TPPII catalytic domain, TPPII encoding nucleic acid or TPPII increasing or activating compound for counteracting and/or preventing a polyglutamine-mediated protein aggregation disorder is also provided. One embodiment thus provides a method for counteracting and/or preventing a polyglutamine-mediated protein aggregation disorder, comprising administering to an individual in need thereof a pharmaceutically effective amount of TPPII, or administering a pharmaceutically effective amount of a functional catalytic domain of TPPII, or administering a pharmaceutically effective amount of a nucleic acid sequence encoding TPPII or a functional catalytic domain of TPPII, or administering a pharmaceutically effective amount of a compound that is capable of enhancing the expression, amount and/or peptidase activity of TPPII. Preferably, said method is performed after said individual has been diagnosed with said disorder associated with polyglutamine-mediated protein aggregation or after said individual has been diagnosed with a risk of developing said disorder.

In yet another embodiment a screening assay is provided. Now that the invention provides the insight that polyglutamine-mediated protein aggregation is counteracted by increasing the expression, amount and/or peptidase activity of a peptidase capable of cleaving a polyQ peptide comprising at least 45 glutamine residues, it has become possible to screen candidate compounds for their effect upon such peptidase and, hence, for their (indirect) capability of counteracting protein aggregation. A compound that is capable of increasing the expression, amount and/or peptidase activity of an (endogenous) peptidase capable of cleaving a polyQ peptide comprising at least 45 glutamine residues will have the overall effect that protein aggregation is diminished. Such compound is therefore suitable for use against protein aggregation, both in vitro and in vivo. Further provided is therefore a method for determining whether a candidate compound is capable of counteracting protein aggregation, the method comprising determining whether said candidate compound is capable of increasing the expression, amount and/or peptidase activity of a peptidase capable of cleaving a polyQ peptide comprising at least 45 glutamine residues. Said method preferably further comprises selecting a candidate compound which is capable of enhancing expression, amount and/or peptidase activity of said peptidase.

It is preferably determined whether a candidate compound is capable of increasing the expression, amount and/or peptidase activity of TPPII. Various methods are available for measuring whether a candidate compound is capable of increasing the expression, amount and/or activity of a peptidase of interest. For instance, a cell comprising a nucleic acid construct comprising a marker gene operably linked to a promoter specific for said peptidase is provided with a candidate compound. Subsequently, expression of said marker gene is measured. If expression of said marker gene appears to be upregulated in cells which are provided with a candidate compound, as compared to cells which are not provided with said candidate compound, it demonstrates that said candidate compound is capable of upregulating expression of said peptidase since the marker gene is operably linked to a promoter specific for said peptidase. In a preferred embodiment, a nucleic acid construct is used which comprises a TPPII-specific promoter operably linked to a marker gene. The promoter of human TPPII is for instance described in Lindas et al, 2007. Any marker gene available in the art is suitable for a test method according to the present invention. Non-limiting examples of marker genes are GFP and luciferase. Preferably, a plurality of candidate compounds is incubated with nucleic acid constructs comprising a TPPII-specific promoter operably linked to a marker gene. Expression of said marker gene is preferably measured both before and after incubation with a candidate compound. If expression of said marker gene appears to be upregulated in cells which are provided with a candidate compound, as compared to cells which are not provided with said candidate compound, it demonstrates that said candidate compound is capable of upregulating TPPII expression since the marker gene is operably linked to a TPPII-specific promoter. This way, a candidate compound capable of enhancing TPPII expression is identified. Such candidate compound is preferably selected. Of course, many alternative test methods are available, which are within the knowledge of the skilled person. For instance, it is known that TPPII is present in cells as an oligomeric complex with a molecular weight of several megaDaltons. TPPII can form different complexes varying in the number of TPPII subunits, which complexes differ in their peptidase activity. Hence, altering TPPII complex formation influences its activity. A screening assay is therefore provided wherein (cellular) TPPII is provided with a candidate compound, where after it is determined whether its peptidase activity has been increased. If the TPPII peptidase activity appears to be increased in cells which are provided with a candidate compound, as compared to cells which are not provided with said candidate compound, it demonstrates that said candidate compound is capable of increasing the peptidase activity of TPPII. This way, a candidate compound capable of enhancing TPPII activity is identified. Such candidate compound is preferably selected. In yet another embodiment, increased activity of TPPII against polyQ peptides is measured using a polyQ peptide flanked on one end by a fluorophore and on the other side by a quencher. Only when TPPII cleaves the polyQ repeat between the fluorophore and the quencher, the quencher is released and fluorescence is measured. This way, too, it is possible to investigate whether a candidate compound is capable of enhancing the activity of TPPII by determining whether or not fluorescence increases after administration of the candidate compound. If a candidate compound is capable of enhancing the activity of TPPII, a faster increase of fluorescence will be detected, as the quenched polyQ peptide is more rapidly degraded. The use of fluorophores and quenchers is a technique well known in the art. This technique is for instance described in Reits et al, Immunity 2003 Jan;18 (1):97-108.

Further provided is therefore a method for determining whether a candidate compound is capable of counteracting protein aggregation, the method comprising determining whether said candidate compound is capable of increasing the expression, amount and/or peptidase activity of TPPII. Said method preferably further comprises comprising selecting a candidate compound which is capable of enhancing expression, amount and/or peptidase activity of TPPII.

In yet another aspect, the invention provides a use of a peptidase, functional catalytic domain, nucleic acid sequence or compound according to the invention for counteracting and/or preventing the formation of aggregating side products during in vitro protein production.

Proteins, such as for instance therapeutic proteins, are often produced in vitro, for instance in prokaryotic or eukaryotic production systems such as Chinese hamster ovary cells, HEK 293 cells, COS-7 cells, HeLa cells, Vero cells, and PER.C6 cells. Furthermore, proteins are produced in whole organisms. Therapeutic proteins are proteins that have been engineered in the laboratory for pharmaceutical use and often comprise a recombinant protein. Therapeutic proteins are used to treat patients suffering from many conditions, including, but not limited to, cancer, Gaucher's disease, diabetes, anaemia, and haemophilia. Major therapeutic proteins comprise monoclonal antibodies, interferon, and erythropoietin.

During in vitro production of proteins of interest, side products are often formed. These side products may aggregate into insoluble intracellular complexes, especially when a protein of interest is produced which comprises a polyQ stretch (either a small or long stretch). When aggregation has initiated, other proteins often become part of the aggregates as well, including the protein of interest. This involves the risks of product loss, loss of biological activity, and enhanced immunogenicity of said product.

Hence, especially when a protein of interest comprises a polyQ stretch, its in vitro production involves the risk of the formation of aggregating side products in the production cells. In such case, a peptidase, functional catalytic domain, nucleic acid sequence or compound according to the invention is preferably used in order to counteract aggregation. This is particularly advantageous when a therapeutic protein is produced in vitro. Other areas that involve in vitro production of proteins, and especially purified proteins, and which will benefit from a reduction of aggregate formation include the food industry, structural proteomics and the development and production of in vitro assays such as enzyme-linked immunoabsorbant assay and protein activity assays.

One embodiment of the invention provides a use of a peptidase, functional catalytic domain, nucleic acid sequence or compound according to the invention for counteracting and/or preventing aggregation of a protein in vitro, whereby counteracting and/or preventing aggregation results in enhanced recovery of said protein. Optimizing the levels of soluble protein is an attractive strategy to increase pure and active protein yield compared to recovering highly expressed protein in aggregated form. Recovery of aggregated proteins is usually poor and often affects the integrity and activity of the recovered protein. In addition, purification of over- expressed soluble proteins is faster and cheaper than obtaining it from aggregated forms.

Alternatively, a peptidase, catalytic domain, nucleic acid sequence or compound according to the invention is used in order to counteract and/or prevent aggregation of a polyQ protein in vivo.

One embodiment of the invention therefore provides a method for counteracting and/or preventing aggregation of a polyQ protein in a cell, comprising providing said cell with a peptidase capable of cleaving a polyQ peptide comprising at least 45 glutamine residues, or a functional catalytic domain of said peptidase, or with a nucleic acid sequence encoding said peptidase or functional catalytic domain, or with a compound that is capable of enhancing the expression, amount and/or peptidase activity of said peptidase. In one embodiment said cell comprises an in vitro protein production cell.

As stated before, said peptidase preferably comprises a cysteine peptidase or a serine peptidase. Most preferably, said peptidase comprises TPPII. Further provided is therefore an in vitro method for counteracting and/or preventing aggregation of a polyQ protein in a cell, comprising providing said cell with TPPII or with a functional catalytic domain of TPPII, or with a nucleic acid sequence encoding TPPII or a functional catalytic domain of TPPII, or with a compound that is capable of enhancing the expression, amount and/or peptidase activity of TPPII.

In yet another embodiment, a peptidase, functional catalytic domain, nucleic acid sequence or compound according to the invention is combined with another compound capable of counteracting polyQ protein aggregation. As disclosed in patent application WO 2008/127100, a heat shock protein which is a member of the Hsp4O/DnaJ family (also called herein a DnaJ heat shock protein) is suitable for counteracting polyQ protein aggregation. Hsp4O/DnaJ heat shock proteins are homologous to the Escherichia coli DnaJ protein and contain a characteristic J domain that mediates interaction with Hsp70 and regulate ATPase activity by Hsp70. According to the present invention, a DnaJ heat shock protein acts in concert with a peptidase according to the invention: the heat shock protein keeps long polyQ peptides in solution, allowing a peptidase according to the present invention to cleave the polyQ peptide. A DnaJ heat shock protein, or at least an aggregation inhibiting part thereof, or a nucleic acid sequence encoding such heat shock protein or aggregation inhibiting part, is therefore preferably combined with a peptidase, functional catalytic domain, nucleic acid sequence or compound according to the invention in order to improve efficiency of inhibition of polyQ protein aggregation. It is also possible to combine a peptidase, functional catalytic domain, nucleic acid sequence or compound according to the invention with a compound that is capable of enhancing the expression, amount and/or protein aggregation inhibiting activity of a DnaJ heat shock protein. Such compound is capable of indirectly keeping a long polyQ peptide is solution by increasing the amount or activity of a DnaJ heat shock protein, thereby allowing a peptide according to the invention to cleave the long polyQ peptide. Hence, a combination of such compound and a peptidase, functional catalytic domain, nucleic acid sequence or compound according to the invention will result in enhanced anti aggregation activity.

A combination according to the invention is suitable for various applications according to the invention, such as the therapeutic applications, testing applications and in vitro protein production applications as described herein before. Further provided is therefore a use or method or peptidase (preferably TPPII) or functional catalytic domain or nucleic acid sequence or compound according to the present invention, wherein a combination is used comprising:
a peptidase capable of cleaving a polyQ peptide comprising at least 45 glutamine residues, or a functional catalytic domain of said peptidase, or a nucleic acid sequence encoding said peptidase or functional catalytic domain, or a compound that is capable of enhancing the expression, amount and/or peptidase activity of said peptidase; and
a DnaJ heat shock protein or a functional protein aggregation inhibiting part thereof, and/or a nucleic acid sequence encoding a DnaJ heat shock protein or a functional protein aggregation inhibiting part thereof, and/or a compound that is capable of enhancing the expression, amount and/or protein aggregation inhibiting activity of a DnaJ heat shock protein.

Said DnaJ heat shock protein preferably comprises DnaJB6 and/or DnaJB8. According to the present invention, DnaJB6 and DnaJB8 are particularly well capable of counteracting aggregation of polyQ peptides. As a result, the polyQ peptides remain soluble during a longer period of time, thereby facilitating their cleavage by a peptidase according to the invention. In view of this, DnaJB6 and/or DnaJB8 are preferably combined with a peptidase, functional catalytic domain, nucleic acid sequence or compound according to the invention in order to improve anti aggregation activity. It is, of course, also possible to use a functional protein aggregation inhibiting part of DnaJB6 or DnaJB8, and/or a nucleic acid sequence encoding DnaJB6 or DnaJB8 or a functional protein aggregation inhibiting part thereof.

Further provided is therefore a use, method, combination or composition according to the invention, wherein said DnaJ heat shock protein comprises DnaJB6 and/or DnaJB8. In one preferred embodiment, DnaJB6 and/or DnaJB8 is combined with TPPII (or with a nucleic acid sequence encoding TPPII). As shown in the Examples, a combination of TPPII and DnaJB8, as well as a combination of TPPII andDnaJB6, is particularly well capable of counteracting polyQ protein aggregation.

In one embodiment, a compound that is capable of enhancing the expression, amount and/or protein aggregation inhibiting activity of DnaJB6 and/or DnaJB8 is used in combination with a peptidase, functional catalytic domain, nucleic acid sequence or compound according to the invention. In this embodiment, a compound is preferably used which is capable of enhancing the expression, amount and/or protein aggregation inhibiting activity of DnaJB6, because DnaJB6 is ubiquitously present in mammals. As disclosed in patent application PCT/NL2009/050235, histone deacetylase-4 (HDAC-4) or a functional part or functional derivative thereof is particularly suitable for increasing anti-protein aggregation activity of DnaJB6 and DnaJB8. Histone deacetylases are enzymes that are known to catalyze the acetylation of proteins at lysine residues. Although originally discovered as histone modification it is nowadays known that many proteins can be post-translationally modified by (de)acetylation, which drastically affects protein stability and function. Since HDAC-4 increases anti-protein aggregation activity of DnaJB6 and DnaJB8, it is preferably combined with a peptidase, functional catalytic domain, nucleic acid sequence or compound according to the invention in order to improve anti aggregation activity. Further provided is therefore a use, method, combination or composition according to the invention, wherein said compound capable of enhancing the protein aggregation inhibiting activity of said DnaJ heat shock protein comprises histone deacetylase-4 (HDAC-4) or a functional catalytic domain of HDAC-4 or a nucleic acid sequence encoding HDAC-4 or encoding a functional catalytic domain of HDAC-4.

A functional part of HDAC-4 is defined herein as a part which has the same properties in kind, not necessarily in amount. A functional part of HDAC-4 also has the capability of enhancing anti protein aggregation activity of Hsp40/DnaJ heat shock proteins, preferably DnaJB8 and/or DnaJB6, albeit not necessarily to the same extent as HDAC-4.

The term “functional derivative of HDAC-4” refers to a modified form of a HDAC-4 protein, including but not limited to a glycosylated form and/or a pegylated form, which may improve the pharmacological properties of a protein drug and may also expand its half life. The term “HDAC-4 derivative” embraces HDAC-4 proteins that are modified such that their functionality is increased and/or that are modified such that they have become more stable as compared to wild type HDAC-4. A functional derivative of HDAC-4 also has the capability of enhancing anti protein aggregation activity of Hsp40/DnaJ heat shock proteins, preferably DnaJB8 and/or DnaJB6, albeit not necessarily to the same extent as HDAC-4.

A preferred combination according to the present invention thus comprises a peptidase, functional catalytic domain, nucleic acid sequence or compound according to the invention together with DnaJB6, DnaJB8 and/or HDAC-4. Alternatively, a functional part of DnaJB6, DnaJB8 and/or HDAC-4 or a nucleic acid encoding DnaJB6, DnaJB8, HDAC-4 is used. Furthermore, as described above, a preferred peptidase according to the present invention is TPPII. A particularly preferred combination according to the present invention therefore comprises:
TPPII or a functional catalytic domain of TPPII, and/or a nucleic acid sequence encoding TPPII or functional catalytic domain of TPPII, and/or a compound that is capable of enhancing the expression, amount and/or peptidase activity of TPPII; and
DnaJB6 or DnaJB8 or a functional protein aggregation inhibiting part thereof, and/or a nucleic acid sequence encoding DnaJB6 or DnaJB8 or a functional protein aggregation inhibiting part thereof, and/or HDAC-4 or a functional catalytic domain of HDAC-4 or a nucleic acid sequence encoding HDAC-4 or encoding a functional catalytic domain of HDAC-4.

This preferred combination is preferably used in various applications according to the invention, such as the therapeutic applications, testing applications and in vitro protein production applications as described herein before. Further provided is therefore a use or method according to the invention, wherein a combination is used comprising:
TPPII or a functional catalytic domain of TPPII, and/or a nucleic acid sequence encoding TPPII or functional catalytic domain of TPPII, and/or a compound that is capable of enhancing the expression, amount and/or peptidase activity of TPPII; and
DnaJB6 or DnaJB8 or a functional protein aggregation inhibiting part thereof, and/or a nucleic acid sequence encoding DnaJB6 or DnaJB8 or a functional protein aggregation inhibiting part thereof, and/or HDAC-4 or a functional catalytic domain of HDAC-4 or a nucleic acid sequence encoding HDAC-4 or encoding a functional catalytic domain of HDAC-4.

The compounds of a combination according to the present invention do not need to be used simultaneously. It is, for instance, possible to administer a first compound, such as a peptidase, functional catalytic domain, nucleic acid sequence or compound according to the invention, to an individual at a different time point than the second compound (such as a DnaJ heat shock protein or a functional protein aggregation inhibiting part thereof or a nucleic acid sequence encoding said DnaJ heat shock protein or functional protein aggregation inhibiting part thereof, or a compound that is capable of enhancing the expression, amount and/or protein aggregation inhibiting activity of said DnaJ heat shock protein). Alternatively, however, the compounds of a combination according to the present invention are used simultaneously.

A combination according to the present invention is particularly suitable for use of a medicament. One embodiment therefore provides a combination of:
a peptidase capable of cleaving a polyQ peptide comprising at least 45 glutamine residues, or a functional catalytic domain of said peptidase, or a nucleic acid sequence encoding said peptidase or functional catalytic domain, or a compound that is capable of enhancing the expression, amount and/or peptidase activity of said peptidase; and
a DnaJ heat shock protein or a functional protein aggregation inhibiting part thereof, and/or a nucleic acid sequence encoding a DnaJ heat shock protein or a functional protein aggregation inhibiting part thereof, and/or a compound that is capable of enhancing the expression, amount and/or protein aggregation inhibiting activity of a DnaJ heat shock protein, for use as a medicament.

As said before, said peptidase preferably comprises TPPII, said DnaJ heat shock protein preferably comprises DnaJB6 and/or DnaJB8 and said compound capable of enhancing the protein aggregation inhibiting activity of said DnaJ heat shock protein comprises histone deacetylase-4 (HDAC-4) or a functional catalytic domain of HDAC-4 or a nucleic acid sequence encoding HDAC-4 or encoding a functional catalytic domain of HDAC-4.

Yet another embodiment provides a pharmaceutical composition comprising a combination according to the present invention. One embodiment therefore provides a pharmaceutical composition comprising:
a peptidase capable of cleaving a polyQ peptide comprising at least 45 glutamine residues, or a functional catalytic domain of said peptidase, and/or a nucleic acid sequence encoding said peptidase or functional catalytic domain, and/or a compound that is capable of enhancing the expression, amount and/or peptidase activity of said peptidase; and
a DnaJ heat shock protein or a functional protein aggregation inhibiting part thereof, and/or a nucleic acid sequence encoding a DnaJ heat shock protein or a functional protein aggregation inhibiting part thereof, and/or a compound that is capable of enhancing the expression, amount and/or protein aggregation inhibiting activity of a DnaJ heat shock protein;
   and, optionally, a pharmaceutical acceptable carrier, diluent or excipient.

A pharmaceutical composition according to the invention may be presented in any form, for example as a tablet, as an injectable fluid or as an infusion fluid etc. Moreover, said pharmaceutical composition can be administered via different routes, for example intravenously, rectally, bronchially, or orally. It is clear for the skilled person, that preferably an effective amount is delivered. The compositions may optionally comprise pharmaceutically acceptable excipients, stabilizers, activators, carriers, permeators, propellants, desinfectants, diluents and preservatives. Suitable excipients are commonly known in the art of pharmaceutical formulation and may be readily found and applied by the skilled artisan.

For oral administration, a pharmaceutical composition according to the invention is, for example, administered in solid dosage forms, such as capsules, tablets (preferably with an enteric coating), and powders, or in liquid dosage forms, such as elixirs, syrups, and suspensions. A pharmaceutical composition according to the invention can be encapsulated in gelatin capsules together with inactive ingredients and powdered carriers, such as glucose, lactose, sucrose, mannitol, starch, cellulose or cellulose derivatives, magnesium stearate, stearic acid, sodium saccharin, talcum, magnesium carbonate and the like. Examples of additional inactive ingredients that may be added to provide desirable colour, taste, stability, buffering capacity, dispersion or other known desirable features are red iron oxide, silica gel, sodium lauryl sulphate, titanium dioxide, edible white ink and the like. Similar diluents can be used to make compressed tablets. Both tablets and capsules can be manufactured as sustained release products to provide for continuous release of medication over a period of hours. Compressed tablets can be sugar coated or film coated to mask any unpleasant taste and protect the tablet from the atmosphere, or enteric-coated for selective disintegration in the gastrointestinal tract. Liquid dosage forms for oral administration can contain colouring and flavouring to increase patient acceptance.

In a preferred embodiment a pharmaceutical composition according to the invention is suitable for oral administration and comprises an enteric coating to protect the composition from the adverse effects of gastric juices and low pH. Enteric coating and controlled release formulations are well known in the art. Enteric coating compositions in the art may comprise of a solution of a water-soluble enteric coating polymer mixed with the active ingredient(s) and other excipients, which are dispersed in an aqueous solution and which may subsequently be dried and/or pelleted. The enteric coating formed offers resistance to attack of the active ingredient(s) by atmospheric moisture and oxygen during storage and by gastric fluids and low pH after ingestion, while being readily broken down under the alkaline conditions which exist in the lower intestinal tract.

As said before, said peptidase preferably comprises TPPII. Moreover, said DnaJ heat shock protein preferably comprises DnaJB6 and/or DnaJB8. Furthermore, said compound capable of enhancing the protein aggregation inhibiting activity of said DnaJ heat shock protein preferably comprises histone deacetylase-4 (HDAC-4) or a functional catalytic domain of HDAC-4 or a nucleic acid sequence encoding HDAC-4 or encoding a functional catalytic domain of HDAC-4. Further provided is therefore a pharmaceutical composition according to the invention, comprising:
TPPII, or a functional catalytic domain of TPPII, and/or a nucleic acid sequence encoding TPPII or functional catalytic domain of TPPII, and/or a compound that is capable of enhancing the expression, amount and/or peptidase activity of TPPII; and
DnaJB6 or DnaJB8 or a functional protein aggregation inhibiting part thereof, and/or a nucleic acid sequence encoding DnaJB6 or DnaJB8 or a functional protein aggregation inhibiting part thereof, and/or HDAC-4 or a functional catalytic domain of HDAC-4 or a nucleic acid sequence encoding HDAC-4 or encoding a functional catalytic domain of HDAC-4;
and, optionally, a pharmaceutical acceptable carrier, diluent or excipient.

Another aspect of the present invention provides an improved polyQ construct. A polyQ construct according to the present invention is, amongst other things, particularly useful for assays wherein aggregation of polyQ proteins is mimicked. More particularly, a polyQ construct according to the invention is particularly useful for mimicking (monomeric) polyQ peptide release by the proteasomes of living cells. A polyQ construct according to the present invention consists of a ubiquitin-polyQ fusion construct wherein ubiquitin is directly linked to a polyQ peptide which consists of glutamine residues only. Upon synthesis in a cell, a ubiquitin-polyQ fusion construct according to the invention is immediately cleaved into ubiquitin (Ub) and a polyQ peptide. The resulting peptide has no flanking amino acids, no tags and no flanking methionine; it represents a pure monomeric polyQ peptide which is released from the proteasome after polyQ protein degradation.

Other studies in this area investigating polyQ disorders use polyQ constructs that contain a starting methionine and/or fusion tags like fluorescent proteins. These polyQ constructs, including polyQ-GFP, huntingtin exon-1 or their short-lived variants, will require processing by the proteasome and therefore do not represent polyQ peptides generated by the proteasome. Hence, the polyQ proteins that are currently used represent proteins upstream of the proteasome, whereas a polyQ construct according to the present invention represents a pure polyQ peptide downstream of the proteasome. In other studies, polyQ peptides with additional amino acids for solubility (not linked to ubiquitin) are administered to the medium of cultured cells. However, such peptides appear to be taken up by the cells as aggregate complexes via endocytosis. Such cellular aggregates still do not represent monomeric polyQ peptides which are released from the proteasome after polyQ protein degradation. Contrary, the present inventors have succeeded in generating cellular polyQ peptides representing monomeric polyQ peptides released from the proteasome. This was possible by using a ubiquitin-polyQ fusion construct according to the invention which is immediately cleaved into ubiquitin (Ub) and a monomeric polyQ peptide after synthesis in a cell. Such ubiquitin-polyQ fusion construct is especially useful in a method for determining whether a candidate compound is capable of counteracting protein aggregation.

Further provided is therefore a ubiquitin-polyQ fusion construct consisting of ubiquitin and a polyQ peptide, wherein said polyQ peptide consists of glutamine residues only. Also provided is a method according to the invention wherein said ubiquitin-polyQ fusion is used. One aspect thus provides a method for determining whether a candidate compound is capable of counteracting protein aggregation, the method comprising determining whether said candidate compound is capable of increasing the expression, amount and/or peptidase activity of a peptidase capable of cleaving a polyQ peptide comprising at least 45 glutamine residues, characterised in that a ubiquitin-polyQ fusion construct is used that consists of ubiquitin and a polyQ peptide, wherein said polyQ peptide consists of glutamine residues only. It is preferably determined whether a candidate compound is capable of increasing the expression, amount and/or peptidase activity of a peptidase capable of cleaving a polyQ peptide derived from said ubiquitin-polyQ fusion construct (preferably after cleavage of said fusion construct into ubiquitin and a monomeric polyQ peptide by a ubiquitin C-terminal hydrolase).

Said ubiquitin-polyQ fusion construct according to the invention is preferably synthesized in cells. To that end, a nucleic acid sequence encoding a ubiquitin-polyQ fusion construct according to the invention is preferably introduced into a cell. A preferred embodiment therefore provides a nucleic acid sequence encoding a ubiquitin-polyQ fusion construct, which construct consists of ubiquitin and a polyQ peptide, wherein said polyQ peptide consists of glutamine residues only. Said nucleic acid sequence according to the present invention is preferably introduced into cells in order to generate intracellular polyQ peptides. Subsequently, it is possible to investigate aggregation. Said polyQ peptide preferably has a length of at least 10 glutamine residues. In one embodiment, said polyQ peptide has a length of about 10-200 glutamine residues. Aggregation of disease-related polyQ proteins is preferably investigated. In one particular embodiment, therefore, said polyQ peptide has a length of at least 45 glutamine residues.

In yet another embodiment, a ubiquitin of a polyQ construct according to the invention is linked to a detectable moiety. This enables visualisation of cleavage of said ubiquitin-polyQ fusion construct into ubiquitin and a polyQ peptide, directly upon synthesis of said construct in a cell. Further provided is therefore a compound comprising a fusion construct according to the invention, wherein said ubiquitin is linked to a detectable moiety. A nucleic acid sequence encoding such compound according to the invention is also provided. In one particularly preferred embodiment, said construct is a GFP-Ub-polyQ construct, wherein GFP labelled ubiquitin is linked to a polyQ peptide which consists of glutamine residues only. As shown in the Examples, this construct is particularly suitable for mimicking aggregation of polyQ proteins. Of course, any other detectable moiety known in the art can be used instead of GFP, such as for instance luciferase.

Also provided is a ubiquitin-polyQ fusion construct according to the invention for use in a method according to the invention for determining whether a candidate compound is capable of counteracting protein aggregation. One aspect thus provides a method for determining whether a candidate compound is capable of counteracting protein aggregation, the method comprising determining whether said candidate compound is capable of increasing the expression, amount and/or peptidase activity of a peptidase capable of cleaving a polyQ peptide comprising at least 45 glutamine residues, characterised in that a ubiquitin-polyQ fusion construct according to the invention or a compound comprising a fusion construct according to the invention or a nucleic acid sequence encoding a fusion construct or compound according to the invention is used

The invention is further explained in the following examples. These examples do not limit the scope of the invention, but merely serve to clarify the invention.

### EXAMPLES

### Example 1

Numerous neurodegenerative diseases are manifested by the accumulation and aggregation of intracellular proteins. These diseases include polyglutamine (polyQ) expansion disorders like Huntington's disease (HD), Spinal Bulbar Muscular Atrophy (SBMA) and various spinocerebellar ataxia's (SCAs). PolyQ disorders are dominantly inherited and caused by expansions of CAG repeats. Normally, the disease-related proteins involved contain sequences of 6-40 glutamine repeats, while expansion of these tracts to 40-300 repeats leads to disease. The age of onset of the disorder is inversely correlated with the repeat length of the polyQ tracts.

The proteasome can degrade both wild-type and expanded forms of most polyQ proteins. Surprisingly, polyQ-expanded proteins are not degraded to completion by the proteasome both in vitro and in vivo. While flanking amino acids may be removed by exo-peptidases, the polyQ tracts themselves accumulate when not efficiently cleared by downstream peptidases.

To examine the fate of these polyQ peptides downstream the proteasome, we mimicked proteasomal generation of polyQ peptides in living cells. Most studies investigating polyQ disorders use polyQ constructs that contain a starting methionine and/or fusion tags like fluorescent proteins. These polyQ constructs, including polyQ-GFP, huntingtin exon-1 or their short-lived variants, will require processing by the proteasome and therefore do not represent polyQ peptides generated by the proteasome. To mimic pure polyQ peptide generation, we generated a fusion protein containing green fluorescent protein, ubiquitin and polyQ peptides (GFP-Ub-polyQ). This fusion protein efficiently releases non-tagged polyQ peptides upon cleavage by ubiquitin C-terminal hydrolases. We show that upon release, only polyQ peptides of disease-related lengths accumulated inside cells, and initiated intracellular protein aggregation. Proteasomes were rapidly sequestered, followed by ubiquitinated proteins, and association of chaperones, as has been observed in various polyQ disorders. Also, various proteins containing either wild-type or expanded polyQ stretches were sequestered. In addition, accumulation of expanded polyQ peptides led to neuronal toxicity.

### Material And Methods

### Plasmid Constructs

Ubiquitin (Ub) was generated by PCR from GFP-Ub (Dantuma et al., 2006) with forward primer 5′-CCCGAGCTCAGATGCAGATCTTCGTGAAG-3′ and reverse primer 5′-CTCGGGCCCTCACCCACCTCTGAGACGG-3′ and ligated into EGFP-C1 (Clonetech). The resulting construct GFP-Ub was again generated by PCR with forward primer 5′-CGCGGATCCATGGTGAGCAAGGGCGAG-3′ and a reverse primer 5′-CGGGAATTCCTGCAGCCCACCTCTGAGACGGAG-3′, and ligated into Ub-x-GFP-Q16/65/112 (Verhoef et al, 2002) where the Ub-x-GFP insert was replaced for GFP-Ub, resulting in GFP-Ub-Q16/Q65/Q122. This procedure was required to remove the restricition site PstI present between GFP and Ub, since PstI was also required for Ub-polyQ ligation. The usage of restriction sites required the presence of some flanking amino-acids, resulting in an N-terminal Leu residue and a Glu-Thr-Ser-Pro-Arg sequence at the C-terminus. GFP was exchanged for mRFP to generate the different RFP-Ub-polyQ fusions. The alternative polyQ peptide lengths of Q33 and Q48 were generated by re-transformation of GFP-Ub-Q65, leading to altered polyQ lengths. Q16-GFP was generated by inserting a Q16 repeat (derived from Ub-M-GFP-Q16) in front of GFP. Htt exon-1 was kindly provided by Ron Kopito, Atx3 by Henry Paulson, AR by Paul Taylor, GFP-Ub, RFP-Ub, Ub-M-GFP-polyQ (used to express GFP-polyQ) and β7-RFP by Nico Dantuma, HSP70-GFP by Harm Kampinga, TBP1 by Rick Morimoto and QBP1-CFP by Yoshitaka Nagai.

### Transfections, Cell-Culture And Toxicity Assay

Human embryonic kidney cells (HEK293T) and Mel JuSo fibroblast cells where cultured in IMDM (GIBCO) supplemented with 10% FCS and penicillin/streptomycin/L-glutamine. The cells where transiently transfected with Fugene6 (Roche) and analyzed at indicated time-points after transfection. Mouse STHdh^{+/+}(Q7) cells (kindly provided by Marcy MacDonald) (Trettel et al, 2000) and N2A neuroblastoma cells were cultured in DMEM supplemented with 10% FCS and penicillin/streptomycin/L-glutamine. Neuronal cells where transiently transfected with Lipofectamine 2000 (Invitrogen). Mouse STHdh^{+/+}(Q7) cells were incubated at 32° C. For toxicity measurements, N2A cells were analyzed by FACS LSRII for GFP fluorescence 24 or 48 hours after transfection, and the percentage of GFP-positive cells was quantified.

### Western Blot

Cytosolic extracts were generated by lysing cells with 0.1% Triton X-100 for 30 minutes at 4° C., and supernatant was used after spinning down the lysate. 20 μg of cytosolic protein lysates were separated by 18% SDS-PAGE and transferred to Protan nitrocellulose membranes. Membranes were blocked in 5% dry milk in TBS containing 0.3% tween and probed with 1:1000 anti-GFP (Molecular Probes), 1:100 anti-Ub (SIGMA) or the anti-Polyglutamine 1C2 (MAB1574, Millipore). Polyclonal Horseradish Peroxidase (HRP) conjugated secondary antibodies, anti-rabbit (Sigma) or anti-mouse (DAKO) were used 1:10.000 to detect the primary antibodies via Lumi-lightPLUS westernblotting substrate (Roche). Preparation of SDS-soluble and SDS-insoluble protein fractions was described before (Carra et al., 2008). Briefly, cells were trypsinized, homogenized, and heated for 10 min at 99° C. in sample buffer (70 mM Tris pH 6.8, 1.5% SDS, 20% glycerol) supplemented with 50 mM DTT 72 hours after transfection. Cell lysates were centrifuged for at least 30 minutes at 14.000 rpm at room temperature. Supernatants were used as SDS-soluble fraction to which 0.05% bromophenol blue was added. Pellets represented SDS-insoluble fractions and were dissolved in 100% formic acid, incubated 30 minutes at 37° C., lyophilized overnight in a speed vac (Eppendorf), and resuspended in a ¼ of the volume of sample buffer containing 0.05% bromophenol blue. Samples were separated on either 18% SDS-PAGE (anti-polyQ), or 12.5% SDS-PAGE (anti-GFP) and further treated as Western blots.

### Fluorescence, Confocal And Electronic Microscopy

HEK293T cells were transfected with indicated constructs and the percentages of aggregates were scored using an inverted fluorescence microscope (Leica DMR). For imaging, Mel Juso cells were transiently transfected with indicated constructs and images where obtained using a confocal microscope (Leica SP2) using a 63× objective. Note that some pictures show ‘over-exposed’ fluorescent aggregates in order to visualize non-sequestered, cytoplasmic staining. For immunostaining, Mel Juso cells were fixed with 4% paraformaldehyde and permeabilized using 0.1% triton in PBS containing 1% FCS and stained with the primary antibodies 1C2 or MW1 (Ko et al, 2001) (1:1000), followed by goat anti-mouse Cy3 labeling (Jackson ImmunoResearch Laboratories). The MW1 antibody developed by Ko, Ou and Patterson was obtained from the Developmental Studies Hybridoma Bank under the auspices of the NICHD and maintained by the University of Iowa. For endogenous Hsp70 labeling, Mel Juso cells were stained against Hsp70/Hsc70 (Calbiochem, 1:200) followed by anti-mouse AlexaFluor 633 (Invitrogen). For electron microscopy, Mel Juso cells were embedded in situ. Preceding fixation, cells were washed briefly in 20 mM PBS (pH 7.4). Fixation was done in a mixture of 4% paraformaldehyde, 1% glutaraldehyde in 0.1 M Phosphate Buffer (pH 7.4) for 60 minutes. After fixation cells were washed in distilled water, osmicated for 60 minutes in 1% OsO4 in water, washed again in distilled water, dehydrated through a series of ethanol baths and embedded in LX-112. After polymerization the plastic was removed and small parts of the epon block containing the cells were prepared for ultra-thin sectioning. Ultra-thin sections were cut, collected on formvar coated grids and stained with uranyl acetate and lead citrate. Sections were examined with a Philips EM-420 electron microscope.

### Filter Retardation Assay

Filter retardation assay was performed as described before (Wanker et al, 1999). Briefly, 72 hours after transfection, HEK293T cells were lysed for 30 minutes on ice in Nondinet P-40 (NP-40) buffer (100 mM TrisHCl, pH 7.5, 300 mM NaCl, 2% NP-40, 10 mM EDTA, pH 8.0, supplemented with complete mini protease inhibitor cocktail (Roche) and phosphatase inhibitor cocktail (Sigma). After centrifugation 15 minutes at 14.000 rpm at 4° C., cell pellets were resuspended in benzonase buffer (1 mM MgCl₂, 50 mM Tris-HCl; pH 8.0) and incubated for 1 hour at 37° C. with 250U benzonase Merck). Reactions were stopped by adding 2× termination buffer (40 mM EDTA, 4% SDS, 100 mM DTT). Aliquots of 30 μg protein extract were diluted into 2% SDS buffer (2% SDS, 150 mM NaCl, 10 mM Tris pH 8.0) and filtered through a 2 μm cellulose acetate membrane (Schleicher and Schuell) pre-equilibrated in 2% SDS buffer. Filters were washed twice with 0.1% SDS buffer (0.1% SDS, 150 mM NaCl, 10 mM Tris pH 8.0) and subsequently blocked in 5% nonfat milk (Protifar Plus, Nutricia) in TBS. Captured aggregates were detected by incubation with 1C2 antibody and further treated like western blots. Alternatively, GFP fluorescence of trapped aggregates was analysed by LAS3000.

### Results

PolyQ-expanded peptides accumulate and induce intracellular aggregates To examine the fate of proteasomal-released polyQ-peptides in living cells, we generated fusion proteins of fluorescently-tagged Ub with polyQ peptides of wild-type and disease-related lengths. Upon expression, the C-terminal polyQ peptide will be efficiently released from GFP-Ub by immediate cleavage via ubiquitin C-terminal hydrolases. As a result, the generated polyQ peptide does not contain a starting methionine residue, which may affect degradation properties due to similarities with the N-terminus of a full-length protein (Bachmair et al, 1986). Also no tags (such as fluorophores or antibody epitopes) were directly attached to the polyQ peptide. As Ub was fluorescently tagged, the fluorescence intensity reflected the amount of generated polyQ peptides. PolyQ peptides of 16, 65 or 112 glutamine residues were fused to GFP-Ub resulting in GFP-Ub-Q16, GFP-Ub-Q65 and GFP-Ub-Q112, respectively (FIG. 1A).

Expression of the different GFP-Ub-polyQ constructs and subsequent release of polyQ peptides were analyzed 48 hours after transfection. Western blot analysis demonstrated the presence of GFP-Ub (36 kDa) in all lanes (FIG. 1B, left panel). In addition, a large Ub conjugate was present, as shown before for GFP-Ub (Dantuma et al, 2006). No additional bands were detected that could represent uncleaved GFP-Ub-polyQ proteins. Efficient cleavage was also observed when the Western blot was analysed for Ub (FIG. 1B, right panel). These results demonstrate that all polyQ peptides were efficiently cleaved from the GFP-Ub protein. Subsequent immunoblotting against polyQ using the antibody 1C2 (Trottier et al, 1995) showed that polyQ peptides were present in the GFP-Ub-Q65 and GFP-Ub-Q112 lanes (FIG. 1C). The mobility on SDS-PAGE of expanded polyQ peptides was different from their calculated molecular weights, as has been observed before for polyQ-containing proteins. Some additional high molecular weight bands were present, which may represent oligomeric complexes. The absence of Q16 peptides in cells expressing GFP-Ub-Q16 demonstrates that small Q peptides are efficiently cleared from the cytoplasm. It is unlikely that small Q peptides are not recognized by the 1C2 antibody, since a Q16-GFP fusion protein was recognized by 1C2 with almost equal efficiency as expanded GFP-polyQ fusions (data not shown). Accumulation of Q65 and Q112 peptides, but not of Q16 peptides, shows that expanded polyQ peptides were not efficiently degraded in living cells. To our knowledge, these peptides represent the first group of peptides that are resistant to degradation.

Since proteolytic protein fragments containing polyQ tracts are more aggregation prone than the full-length protein, we examined whether the accumulation of Q65 and Q112 peptides initiated aggregate formation. We observed a similar intracellular distribution of GFP-Ub in cells transfected with either GFP-Ub or GFP-Ub-Q16. GFP-Ub was enriched in the nucleus but was also present in the cytoplasm and on vesicles (FIG. 1D), similar to the distribution of endogenous Ub. In contrast, expression of GFP-Ub-Q65 and GFP-Ub-Q112 resulted in the appearance of a distinct intracellular structure decorated with fluorescent Ub in a high percentage of the transfected cells, present in either the nucleus or cytoplasm. The number of cells containing these structures increased both in time and with polyQ length (FIG. 1E). To see whether the length-dependency of aggregate formation would also hold true for polyQ lengths nearby the threshold, we expressed GFP-Ub fused to polyQ peptides of 33 or 48 glutamine residues. Whereas GFP-Ub-Q33 showed no aggregates, cells expressing GFP-Ub-Q48 showed aggregates, although in a lower percentage of cells when compared to Q65 or Q112 peptides (data not shown). GFP-Ub fluorescence was usually present in a ring around a dark core indicating that Ub was recruited (FIG. 1F). At the ultrastructural level, this structure showed a radiating dense core similar to aggregates formed by non-cleavable GFP-polyQ fusion proteins (FIG. 1F) and expanded huntingtin (Qin et al, 2004). In cells expressing Q65 and Q112 peptides, these dense structures were resistant to SDS and selectively trapped in a filter- retardation assay (Wanker et al, 1999). Immunostaining using 1C2 showed that the trapped structures contained polyQ peptides (FIG. 1G), similar to huntingtin exon-1 Q103 (httex1-Q103-GFP) (Wanker et al, 1999). This shows that expanded polyQ peptides induce intracellular SDS-resistant aggregates. Whereas Httex1-GFP is also positive for GFP, no GFP is present on filter trap with the GFP-Ub-polyQ constructs, demonstrating efficient cleavage of the GFP-Ub-polyQ fusion proteins (FIG. 1G). Also analysis of the soluble and insoluble fraction of cell lysates showed no uncleaved GFP-Ub-Q112 fusion proteins in either fraction (data not shown).

To confirm the presence of polyQ peptides in intracellular aggregates, we immunostained cells expressing Q16, Q65 or Q112 peptides with 1C2. As expected, no polyQ peptides were detected in cells expressing GFP-Ub or GFP-Ub-Q16 (FIG. 1H). However, cells transfected with GFP-Ub-Q65 or GFP-Ub-Q112 showed two patterns of polyQ staining, dependent on the presence of aggregates. When aggregates were not present, cells showed a cytoplasmic polyQ staining, whereas GFP-Ub localization was predominantly nuclear. On the other hand, cells containing polyQ peptide aggregates were not recognized by 1C2 (FIG. 1H, arrows indicate an aggregate). A similar difference in immunostaining was obtained using the anti-polyQ antibody MW1 (Ko et al, 2001) (data not shown). The absence of polyQ staining in cells containing aggregates is thought to be due to the dense aggregate structure and its surrounding protein layers that may shield the polyQ core. Indeed, pretreatment with proteinase K degraded shielding proteins and resulted in positive immunostaining of polyQ peptide aggregates (FIG. 1I), as has been observed previously for huntingtin aggregates (Qin et al, 2004). To further confirm that the aggregates contain polyQ peptides, we used a cyan fluorescent protein (CFP) tagged Q-binding peptide (QBP-1) which selectively binds to polyQ aggregates (Nagai et al, 2000). QBP-1 showed a cytoplasmic distribution pattern when expressed alone or together with RFP-Ub or RFP-Ub-Q16 (data not shown). However, cells harboring aggregates initiated by Q112 peptides showed binding of QBP-1 to aggregates (FIG. 1J). Taken together, these results show that expanded polyQ peptides are not efficiently degraded and subsequently initiate formation of aggregates that display all characteristics of disease-related polyQ aggregates.

### PolvQ Peptide Aggregates Recruit Proteasomes, Ubiquitin And Chaperones

Aggregates formed by expanded polyQ proteins often sequester proteins involved in the ubiquitin proteasome system (UPS) but also chaperones. We examined whether aggregates induced by expanded polyQ peptides showed a similar sequestration of UPS components. GFP-Ub was present in a ring around the aggregates (FIG. 1F). Absence of Ub in the aggregate core can be explained by the lack of lysine residues in polyQ peptides, thereby excluding ubiquitination of the polyQ peptides. The presence of GFP-Ub around the core was not due to inefficient cleavage of GFP-Ub-polyQ, since no uncleaved GFP-Ub-polyQ fusions could be detected by SDS-PAGE (FIG. 1B) and filtertrap (FIG. 1G). In addition, co-expression of GFP-Ub with RFP-Ub-Q112 showed a similar sequestration of both fluorescently-tagged Ub proteins into aggregates (FIG. 2A), demonstrating efficient cleavage. This shows that the presence of GFP-Ub is due to ubiquitination of sequestered proteins.

We examined whether proteasomes co-localized with polyQ aggregates in our model, by co-expressing the different RFP-Ub-polyQ constructs with GFP-tagged immuno-proteasomal subunit LMP2. LMP2 is efficiently incorporated into active proteasomes (Reits et al, 1997). Notably, LMP2-GFP was present in the core of polyQ aggregates, demonstrating that proteasomes were recruited to aggregates even before Ub sequestration (FIG. 2B). A similar recruitment was observed when using the constitutive proteasome subunit β7 (FIG. 2D). This finding reflects a proteasomal attempt to degrade accumulating polyQ peptides. The sequestered proteasomes and Ub seemed irreversibly trapped, which was revealed when Fluorescence Recovery After Photobleaching (FRAP) (Reits and Neefjes, 2001) was applied to determine on/off rates of the sequestered molecules. Upon photobleaching of one half of an aggregate, no exchange between the sequestered proteasomes or Ub and the surroundings was observed (data not shown). This demonstrates that the proteasome becomes immobilized, as has been previously observed.

We also examined whether chaperones like Hsp70 were bound to polyQ aggregates, as has been observed in polyQ diseases. Upon co-transfection of the different RFP-Ub-polyQ fusion proteins with GFP-tagged Hsp70, we observed an additional ring-like structure of Hsp70-GFP around the Ub-positive aggregate (FIG. 2C). FRAP analysis revealed that Hsp70 was not irreversibly trapped in the aggregate (data not shown), consistent with previous observations. To compare the composition of aggregates initiated by Q112 peptides with aggregates formed by polyQ-expanded huntingtin exon-1, cells were transfected with either GFP-Ub-Q112 or httex1-Q103-GFP together with β7-RFP-tagged proteasomes, and cells were subsequently immunostained for endogenous Hsp70. Triple color analysis showed that the core of the aggregate was positive for proteasomes. This core was surrounded by Ub or httex1-Q103 . Finally, Hsp70 was present within the most outer layer of the aggregate (FIG. 2D). This shows that various proteins associate at different stages or with different affinities during aggregate formation. The presence of GFP-Ub and httex1-Q103 in a similar layer indicates the recruitment of ubiquitinated proteins and polyQ proteins in this stage of aggregate formation. Since aggregates initiated by expanded polyQ peptides also contained Ub, proteasomes and chaperones as has been described before, our model faithfully mimics aggregate formation in polyQ diseases.

### Sequestering of Glutamine-Containing Proteins Into PolyQ Peptide Aggregates

The presence of httex1-Q103 in ring-like structures around the aggregate and not within the core (FIG. 2D) indicates recruitment of large polyQ fragments into aggregates in a later stage. To examine this, we co-expressed RFP-Ub-Q112 and httex1-Q103-GFP. Indeed, we found that httex1-Q103-GFP was sequestered into aggregates induced by polyQ peptides (FIG. 3A). In addition, the aggregation rate of httex1-Q103-GFP was also dramatically increased when Q112 peptides were present (FIG. 7B), which demonstrates that polyQ peptides initiate aggregates that accelerate huntingtin aggregation. Similar results were obtained with truncated polyQ-expanded ataxin-3 (Atx3-Q85-GFP) and the SBMA-related truncated androgen receptor with a Q84 repeat (AR-Q84-GFP) (data not shown).

Aggregates induced by disease-related polyQ proteins also sequester the wild-type protein expressed by the non-expanded allele. We examined whether polyQ peptide aggregates also sequester non-expanded, wild-type polyQ proteins. The non-expanded httex1-Q25-GFP remained freely distributed in cells that co-expressed either RFP-Ub or RFP-Ub-Q16 (data not shown). In contrast, httex1-Q25-GFP was recruited into polyQ peptide aggregates when co-transfected with RFP-Ub-Q112 (FIG. 3B). A similar entrapment of wild-type truncated ataxin-3 (Atx3-Q28-GFP) and the truncated androgen receptor (AR-Q19-GFP) was observed (data not shown). This sequestration of wild-type polyQ proteins therefore leads to loss of function. Sequestering of non-expanded polyQ proteins was not limited to disease-related proteins, as other polyQ proteins were recruited into aggregates initiated by polyQ peptides, including Q16-GFP (FIG. 3C), but also the Q-stretch containing transcription factor TBP1 when nuclear aggregates were present (FIG. 3D).

### PolyQ Peptides Induce Aggregates And Toxicity In Neuronal Cells

To examine whether polyQ peptides also initiate aggregate formation in neuronal cells, we transiently transfected N2A neuroblastoma cells with the various GFP-Ub-polyQ constructs. N2A cells transfected with either GFP-Ub-Q65 or GFP-Ub-Q112 developed aggregates similar to those present in non-neuronal cells (FIG. 4A), whereas GFP-Ub-Q16 expressing cells showed an Ub distribution comparable to GFP-Ub alone. Since HD mostly affects striatal cells, we also used immortalized SThdh^{+/+} striatal cells (Trettel et al, 2000) which similarly generated intracellular aggregates when transfected with GFP-Ub-Q65 or Q112 (FIG. 4A). Many cells rounded up after expression of expanded polyQ peptides, suggesting toxicity, although this did not correlate with the presence of GFP-Ub positive aggregates. To determine whether the expressed polyQ peptides were toxic, the viability of transfected N2A cells was tested using propidium iodide (PI). Expression of expanded polyQ peptides resulted in increased numbers of PI-positive cells (data not shown). However, hardly any double-positive cells were observed. This is explained by the fact that uptake of PI into polyQ peptide expressing cells was often preceded by loss of GFP fluorescence (FIG. 4B) as observed before. Since loss of fluorescence seemed to be associated with cell death, we used another approach to quantify polyQ peptide induced toxicity. To determine changes in the number of GFP-Ub positive cells in time, we used FACS analysis and compared cell populations expressing the different GFP-Ub-polyQ proteins at 24 and 48 hours after transfection. There was no difference in GFP-Ub fluorescence between cells expressing either GFP-Ub or GFP-Q16 in time. However, a significant decrease in fluorescence was observed in cells expressing GFP-Ub-Q112 when compared to GFP-Ub or GFP-Ub-Q16 (p<0.05), demonstrating that expression of Q112 peptides induced cell death (FIG. 4C). GFP-Ub-Q65 had a mild, although not significant, effect on cell death. Taken together, these results show that expanded polyQ peptides form aggregates and become toxic to neuronal cells.

### Discussion

We mimicked intracellular proteasomal polyQ peptide generation as closely as possible by fusing pure polyQ peptides to GFP-tagged Ub. Expression of our constructs resulted in the efficient release of “naked” polyQ peptides due to immediate cleavage by Ub C-terminal hydrolases. This was shown both by SDS-PAGE that showed a band at equal height for GFP-Ub irrespective of the original construct (FIG. 1B), analysis of the filtertrap assay (FIG. 1G) and different intracellular locations of GFP-Ub and polyQ peptides (FIG. 1H). Since the released polyQ peptides do not contain a starting methionine or additional tags, they closely resemble peptide generation by the proteasome. All previous studies have relied on expression of polyQ fusions that did include such features, which can significantly alter the in vivo behavior of polyQ fragments. Starting methionines will lend the peptides resemblance to the N-terminus of proteins, possibly affecting the rate of degradation. Fluorescent tags contain lysine residues, which can serve as targets for ubiquitination and subsequent degradation by the proteasome. The intracellular release of monomeric polyQ peptides is also closer to the in vivo situation than the addition of synthesized polyQ peptide aggregates to cells (Yang et al, 2002).

We showed that only polyQ peptides of disease-related lengths accumulated in the cell and initiated aggregation. The characteristics of aggregates induced by expanded polyQ peptides were similar to aggregates initiated by expression of expanded polyQ-containing proteins. These characteristics include sequestration of proteasomes, ubiquitin and other polyQ containing proteins such as TBP, and the presence of Hsp70. We show here that ‘proteasomal-derived’ expanded polyQ peptides by themselves are sufficient to accumulate and initiate aggregation. Accumulation of expanded polyQ peptides is toxic to neuronal cells.

Based on our findings, we propose a model in which expanded polyQ peptides are degradation-resistant, and their accumulation leads to intracellular polyQ aggregates (FIG. 5). Proteasomes are rapidly recruited into the polyQ core in a final attempt to degrade the expanded polyQ peptides. Subsequently, other proteins are sequestered and ubiquitinated, perhaps due to (partial) unfolding. These events also lead to the binding of chaperones like Hsp70 that recognize denatured proteins. All these events result in concentric ring-like structures formed around the aggregate (FIG. 5). Essential proteins are depleted from the cell, contributing to cellular dysfunction. We conclude that polyQ peptides are fundamental in initiating aggregation and sequestration of different types of proteins including polyQ proteins. We were able to detect expanded polyQ peptides containing Q65 or Q112 on Western blot and by immunostaining in fixed cells, but we were unable to detect any Q16 peptides. These short polyQ peptides are most likely rapidly degraded by downstream peptidases like PSA (Bhutani et al, 2007) that can digest short polyQ peptides.

### Example 2

### Abstract

Several neurodegenerative disorders, including Huntington's disease, are caused by expansion of the polyglutamine (polyQ) tract over 40 glutamines in the disease-related protein. In vitro data demonstrate that proteasomes release pure polyQ peptides upon degradation of proteins containing polyQ tracts. We expressed pure polyQ peptides of variable lengths in living cells to mimic polyQ peptide generation by the proteasome. Only polyQ peptides of disease-related lengths accumulated and initiated aggregation and toxicity. According to our model, these aggregating polyQ peptides represent a common mechanism in different polyQ disorders. We therefore used this model to examine the role of peptidases and chaperones to improve clearance of these toxic fragments via either peptidases or autophagy. When examining different members of the chaperone Hsp40-family, we observed that these chaperones were able to suppress aggregation of expanded polyQ peptides drastically. This shows that these chaperones act at the level of aggregation-initiating polyQ fragments. These polyQ peptides were targeted by serine and cysteine peptidases, with TPPII as one example of a polyQ peptide degrading peptidase.

### Introduction

The common view in the field of polyQ protein aggregation and toxicity has been that large fragments of polyQ proteins that are generated by proteases such as caspases are the initiators of aggregation as observed in all polyQ disorders. These aggregates were also thought to block the proteasome (FIG. 6A). However, recent data showed that the proteasome is not impaired in polyQ disorders, and is able to degrade polyQ proteins, but not the actual polyQ tract. The resulting polyQ peptides are released and initiate aggregation when of disease-related lengths (FIG. 6B). Using Ub-polyQ constructs to mimic polyQ peptide release (as described in example 1), we examined whether particular peptidases target these expanded polyQ peptides, thereby delaying or preventing polyQ aggregation. Since peptidases can only handle monomeric peptides, we also examined whether particular chaperones could assist by preventing polyQ peptide aggregation, allowing clearance by peptidases.

### Material & Methods

### Plasmids

GFP-Ub-polyQ constructs were generated as described in example 1. The different TPPII constructs were kindly provided by Birgitta Tomkinson, Uppsala University, Sweden. The DnaJB constructs were kindly provided by Harm Kampinga, University of Groningen, The Netherlands.

### Western Blot

Cytosolic extracts were generated by lysing cells with 0.1% Triton X-100 for 30 minutes at 4° C., and supernatant was used after spinning down the lysate. 20 μg of cytosolic protein lysates were separated by 18% SDS-PAGE and transferred to Protan nitrocellulose membranes. Membranes were blocked in 5% dry milk in TBS containing 0.3% tween and probed with anti-Polyglutamine 1C2 (MAB1574, Millipore). Preparation of SDS-soluble and SDS-insoluble protein fractions was described before (example 1 and Carra et al., 2008). For peptidase inhibitory studies, transfected cells were treated for eight hours with different peptidase inhibitors before lysates were generated for Western blotting.

### Results

### TPPII Peptidase Is Capable of Degrading PolyQ Peptides

Large polyQ-peptides are preferably cleaved by endo-peptidases, as a few cleavages will result in smaller polyQ peptides below the disease-threshold that can be targeted by recycling amino-peptidases. To measure endo-peptidase activity against glutamines, we designed a probe containing 8 glutamines, flanked by a fluorophore (left) and a quencher (right). When the glutamines are cleaved, the quencher is released from the fluorophore, leading to fluorescence. Since the termini themselves are protected against exo-peptidases, only endo-peptidases can handle the peptide by cleaving the Q stretch. One of the candidate peptidases tested was TPPII, which is a large peptidase complex that shows both endo- and exo-peptidase activity. Inhibition of TPPII reduced degradation of the short polyQ probe, demonstrating that TPPII is capable of cleaving glutamine stretches by endo-peptidase activities (data not shown). Since this experiment was performed with a small Q peptide, we next examined whether TPPII was able to prevent accumulation and aggregation of polyQ peptides derived from GFP-Ub-Q65. Expression of polyQ peptides of 65 glutamines followed by immunoblotting against polyQ using the antibody 1C2 (Trottier et al, 1995) showed that polyQ peptides were present in the GFP-Ub-Q65 lane (FIG. 7, left). Some additional high molecular weight bands were present, representing oligomeric complexes. Expression of human or mouse TPPII resulted in a decrease in both aggregated and low-molecular weight Q65 peptides, demonstrating that TPPII is capable of preventing polyQ peptide accumulation by degrading the peptide. When testing two TPPII variants that have an altered conformation and lowered endo- peptidase activity, the levels of polyQ peptides (and resulting aggregates) increased again, showing that TPPII complex formation affects polyQ peptide degradation. To examine whether TPPII inhibition led to accumulation of polyQ peptides, cells expressing GFP-Ub-Q65 and reduced, respectively increased, TPPII activity were used. This was achieved by either co-expressing TPPII or adding the specific inhibitor of TPPII, butabindide. Whereas TPPII overexpression reduced polyQ levels, inhibition of TPPII led to accumulation of polyQ peptides (FIG. 10, panel A). Similarly, polyQ fragments generated by the proteasome upon degradation of polyQ expanded proteins were targeted by TPPII. When the proteasome was inhibited, polyQ-expanded ataxin-1 was not degraded into polyQ fragments (indicated by arrows). However, these fragments accumulated when TPPII was inhibited, demonstrating that TPPII targets polyQ fragments generated by the proteasome (FIG. 10, panel B).

### DnaJB Proteins Reduce Aggregation PolyQ Peptides

Since peptidases like TPPII can handle only monomeric peptides, we searched for chaperones that prevent or slow down aggregation. As disclosed in patent application WO 2008/127100, two members of the Hsp40 family, DnaJB6 and DnaJB8, were the most potent suppressors of huntingtin (Htt) aggregation. To examine whether these chaperones only prevented sequestration of Htt into aggregates, or whether they are also capable of affecting polyQ peptide aggregation, we examined whether these chaperones also suppressed aggregation of expanded polyQ peptides. Co-expression of DnaJB6 or DnaJB8 together with GFP-Ub-Q65, resulted in a considerable decrease in aggregation. Normally, about 60% of cells expressing Q65 peptides contained aggregates after 72 hours. However, co-expression of the various chaperones reduced the aggregation to less than 20% (data not shown). To see whether the reduction in aggregates also resulted in less SDS-insoluble material, cells expressing GFP-Ub-polyQ with the various chaperones were separated into SDS-soluble and SDS- insoluble fractions. Samples were loaded on SDS-PAGE and subsequently stained against polyQ. Expression of Q65 peptides showed polyQ positive bands in both soluble and insoluble fractions (FIG. 8A). Co-expression of DnaJB6 and DnaJB8 remarkably reduced the amount of polyQ peptides present in the SDS-insoluble fraction, as was expected from the scorings data. Furthermore, expression of DnaJB6 also resulted in a marked decrease of polyQ peptides in the SDS-soluble fraction (FIG. 8A and B). So, besides reducing aggregation of polyQ expanded proteins, DnaJB6 and DnaJB8 are also able to suppress aggregation of expanded polyQ peptides.

To elucidate which clearance pathway led to the degradation of the polyQ peptides, several inhibitors were used in combination with DnaJB6 and DnaJB8. Cysteine proteases were inhibited by E-64, aspartyl proteases by pepstatin A, metalloproteases by bestatin, serine proteases by phenylmethylsulfonyl (PMSF), proteasomes were inhibited by MG-132 and macroautophagy was inhibited by 3-methyladenine (3-MA). Inhibition of cysteine (FIG. 8B, lane 4) and serine (FIG. 8B, lane 6) peptidases led to the re-appearance of polyQ peptides despite the solubilizing actions of DnaJB6, demonstrating that these peptidases are able to degrade polyQ peptides, especially when chaperoned by members of the DnaJB family (FIG. 9).

### BRIEF DESCRIPTION OF THE DRAWINGS

FIG. 1. PolyQ-expanded peptides induce intracellular aggregates. (A). Schematic representation of GFP-Ub-polyQ (Q16, Q65 and Q112) fusion proteins and the generation of polyQ peptides upon synthesis and cleavage by Ub C-terminal hydrolases. (B). Cytosolic cell lysates of HEK293T expressing the different GFP-Ub-polyQ fusions were immuno-blotted against GFP (left) or Ub (right) 48 hours after transfection. GFP-Ub migrated at the same height for all three fusion proteins, indicating efficient cleavage of polyQ from GFP-Ub. Transfection efficiencies were lower for expanded polyQ peptide constructs. (C). Subsequent staining with an antibody against polyQ (1C2) revealed only the presence of polyQ peptides in cells expressing GFP-Ub-Q65 and GFP-Ub-Q112, and not of GFP-Ub-Q16. The asterix indicates potential oligomeric structures. (D) Confocal images of GFP-Ub and the various GFP-Ub-polyQ distribution in Mel JuSo cells. GFP-Ub-Q16 showed a Ub distribution similar to free GFP-Ub, whereas a high percentage of cells expressing GFP-Ub-Q65 and GFP-Ub-Q112 showed Ub redistribution into aggregates. Scalebar˜5 μm. (E) Percentage of transfected HEK293T cells exhibiting fluorescent aggregate at 48 and 72 h after transfection of cells (data are mean±SEM of 3 different experiments). The amount of aggregates in cells expressing expanded polyQ peptides increased both in time and with polyQ length. (F). GFP-Ub was present in a ring around the aggregate induced by GFP-Ub-Q112 (left panel) that had a fibrillar structure at the ultrastructural level (middle panel), similar to structures induced by non-cleavable GFP-Q65 (right panel). Scalebar˜1 μm. (G). Filter retardation assay showed entrapment of aggregates in HEK293T cells expressing GFP-Ub-Q65, GFP-Ub-Q112 and httex1-Q103-GFP, after immunostaining using the 1C2 antibody. In contrast, GFP is only present when the non-cleavable fusion protein Htt-exon1-GFP is used (H). Confocal images of cells expressing GFP-Ub or the various GFP-Ub-polyQ constructs after immunostaining using antibodies against polyQ (1C2). Mel Juso cells expressing GFP-Ub or GFP-Ub-Q16 showed no polyQ staining. Cells expressing GFP-Ub-Q65 and GFP-Ub-Q112 showed cytoplasmic polyQ staining when no aggregates were present. The presence of aggregates depleted the cells of free polyQ peptides, preventing polyQ staining. The arrows indicate an aggregate. Scalebar˜5 μm. (I) Protease K treatment dissolved the protein shells around the polyQ aggregate, resulting in labeling the outside of the aggregation core with the anti-polyQ antibody 1C2. Scalebar˜5 μm. (J). The Q-binding peptide QBP1-CFP was redistributed into aggregates induced by RFP-Ub-Q112. The arrow indicates the presence of a visible aggregate by phase contrast. Scalebar˜5 μm.

FIG. 2. PolyQ peptide aggregates recruit UPS components and chaperones. Mel Juso cells were transfected with the indicated constructs and imaged 48 hours after transfection. (A). Co-expression of GFP-Ub and RFP-Ub derived from RFP-Ub-Q112 resulted in identical redistribution into aggregates. (B). Proteasomes labeled with LMP2-GFP colocalize with the core of aggregates induced by RFP-Ub-Q112, with RFP-Ub surrounding the core. LMP2-GFP was freely distributed in nucleus and cytoplasm of cells expressing RFP-Ub-Q16. (C). The chaperone Hsp70-GFP was redistributed into aggregates induced by RFP-Ub-Q112, and formed an additional ring around the Ub-positive polyQ peptide aggregate. (D). Upon transfection with GFP-Ub-Q112 or httex1-Q103-GFP together with the proteasomal subunit □7-RFP, cells were immunostained for endogenous Hsp70. The proteasome was within the aggregate core, surrounded by Ub and an additional ring of chaperones. Scalebar˜5 μm.

FIG. 3. Sequestration of glutamine-containing proteins into polyQ peptide aggregates. Mel Juso cells were transfected with the indicated constructs and imaged 48 hours after transfection. (A) Expression of RFP-Ub-Q112 led to the sequestering of httex1-Q103-GFP into polyQ aggregates. (B) httex1-Q25-GFP became sequestered into polyQ peptide aggregates when cells were co-transfected with RFP-Ub-Q112. (C) The non-cleavable fusion protein Q16-GFP was diffusely distributed in cytoplasm and nucleus of cells expressing RFP-Ub-Q16, but co-localizes with aggregates induced by RFP-Ub-Q112. (D) The Q-containing transcription factor TBP1 was recruited to aggregates induced by RFP-Ub-Q112 peptides, but only when the polyQ peptide aggregate was nuclear localized. Scalebar˜5 μm.

FIG. 4. PolyQ peptides induce aggregates and toxicity in neuronal cells. (A). Confocal images of N2A neuroblastoma (upper panel) and immortalized SThdh^{+/+} striatal cells (lower panel) showed diffuse cytoplasmic, nuclear and vesicular distribution of ubiquitin in cells expressing GFP-Ub or GFP-Ub-Q16. GFP-Ub was sequestered into aggregates when cells were transfected with GFP-Ub-Q65 or GFP-Ub-Q112. (B). Loss of GFP-Ub coincides with cell death induced by GFP-Ub-Q112 expression as visualized by PI uptake. Timescale is 30 minutes between images taken by automated fluorescence microscopy. (C). Loss of GFP-positive cells was determined by FACS analysis 24 (red) or 48 (blue) hours after transfection (mean±SEM of 3 different experiments, each in triplicate). GFP-Ub-Q112 showed a significant increase in neurotoxicity in N2A neuroblastoma cells when compared to GFP-Ub or GFP-Ub-Q16 (p<0.05, two-tailed unpaired t-test). Scalebar˜5 μm.

FIG. 5. Model of polyQ peptide aggregate formation and sequestering of UPS components. Upon proteasomal degradation of polyQ proteins, pure polyQ peptides are released into the cytoplasm, where peptidases should recycle them into amino acids. Expanded polyQ peptides show resistance to degradation, leading to accumulation and initiation of aggregate-formation. Proteasomes are rapidly recruited in an attempt to degrade the fragments. In time, other proteins including various polyQ proteins are irreversibly sequestered, which become subsequently ubiquitinated. Finally, chaperones like Hsp70 are recruited, possibly as sequestered proteins become partly unfolded.

FIG. 6. Model of polyQ protein processing and aggregation. (A). The general idea in literature is that polyQ proteins are cleaved by proteases like caspases to generate protein fragments containing the polyQ tract, and that these fragments initiate aggregation and impairment of the proteasome. (B). In our model both polyQ proteins and their fragments are efficiently degraded by the proteasome, which results in the release of polyQ peptides. These peptides initiate aggregation and toxicity, and sequester in time polyQ proteins and fragments including commonly used GFP-tagged polyQ proteins.

FIG. 7. TPPII degrades polyQ peptides. Expression of GFPUb-polyQ65 results in the release of monomeric Q65 peptides that initiate aggregation as visualised by Western blotting against polyQ. The lower box represents low-molecular polyQ65, most likely monomeric Q65, whereas aggregates are present in the upper box. Both aggregated and non-aggregated levels of Q65 decrease when TPPII is co-expressed, demonstrating that TPPII targets polyQ peptides. This effect is decreased when the conformation of TPPII is altered (indicated with variant 1 and 2).

FIG. 8. DnaJB 6 and DnaJB8 prevent polyQ peptide aggregation. (A). Expression of DnaJB6 and DnaJB8 reduced insoluble levels of polyQ65 peptides, whereas soluble levels were mostly affected by DnaJB6 (quantified in the right panel). (B). The effect of DnaJB6 on reducing polyQ65 peptide accumulation was prevented when either cysteine or serine peptidases were inhibited, demonstrating that these peptidases are responsible for degrading soluble Q65 peptides.

FIG. 9. Model of polyQ peptide degradation by peptidases, thereby chaperoned by chaperones like DnaJB6 and DnaJB8 that slow down aggregation. If unsuccessful, polyQ peptides will accumulate and form aggregates that are toxic to cells, leading to neurodegeneration.

FIG. 10. TPPII targets polyQ fragments. (A) TPPII expression reduces polyQ peptide levels, whereas inhibition of TPPII increases polyQ peptide levels demonstrating that TPPII activity targets polyQ peptides for degradation and clearance. (B). PolyQ proteins like polyQ-expanded ataxin-1 are degraded by the proteasome, resulting in polyQ fragments (indicated by arrows). Inhibition of the proteasome prevents polyQ fragment generation, whereas these fragments accumulate when TPPII is inhibited showing that these polyQ fragments are cleared by TPPII.

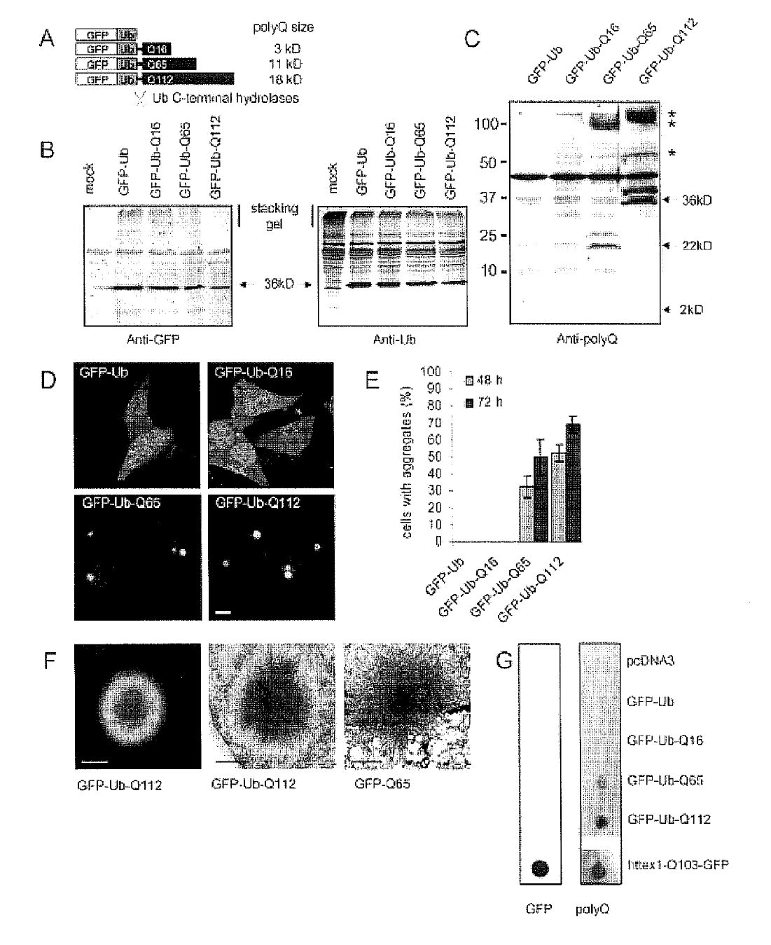

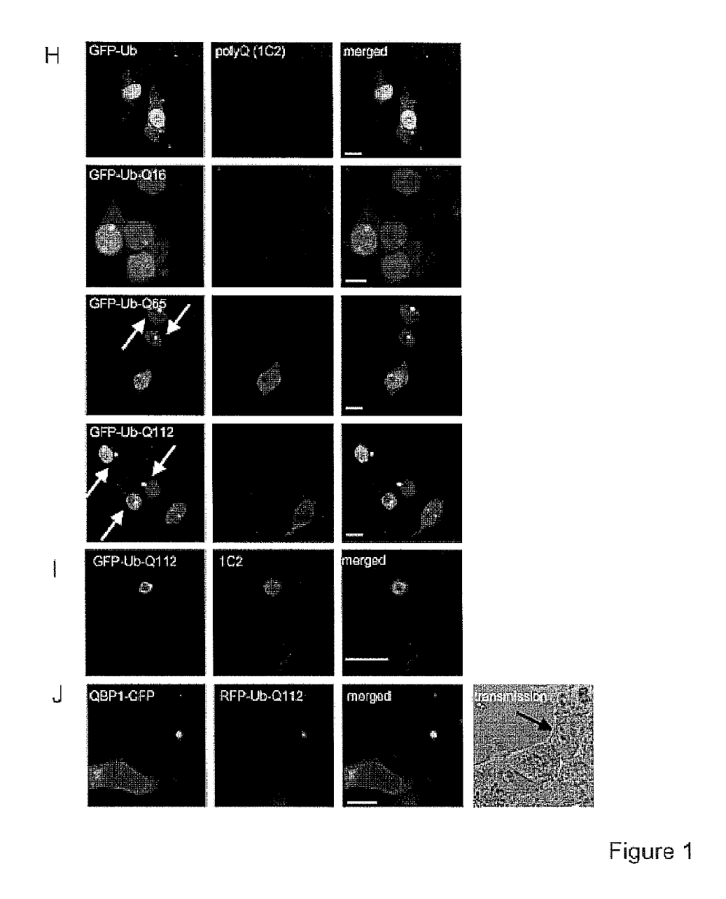

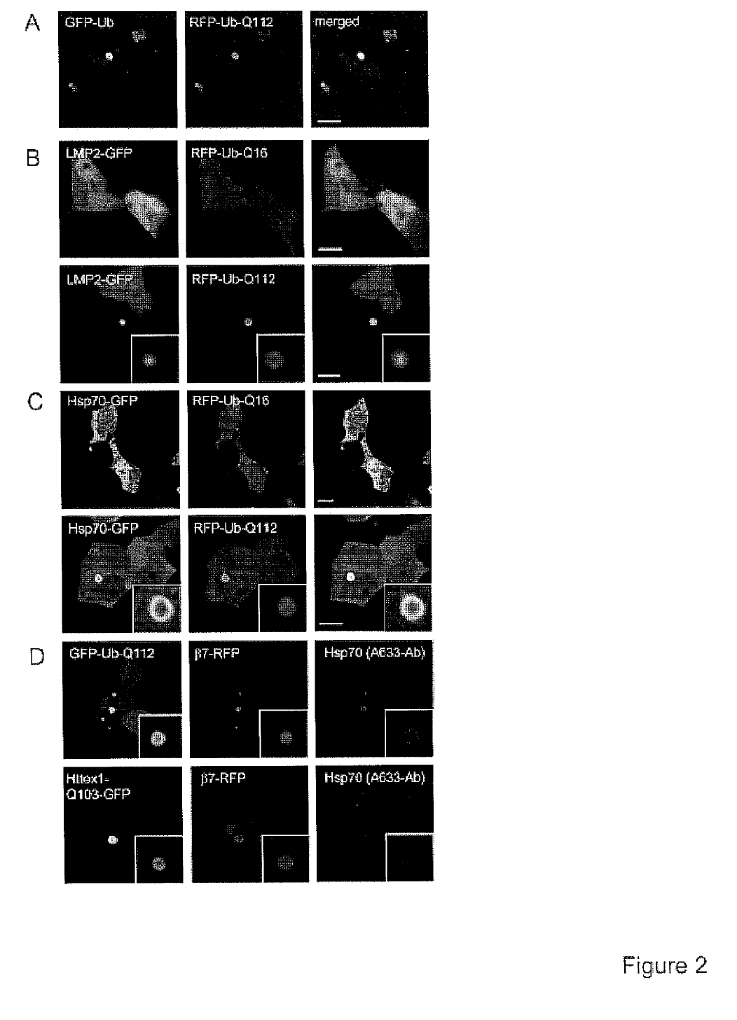

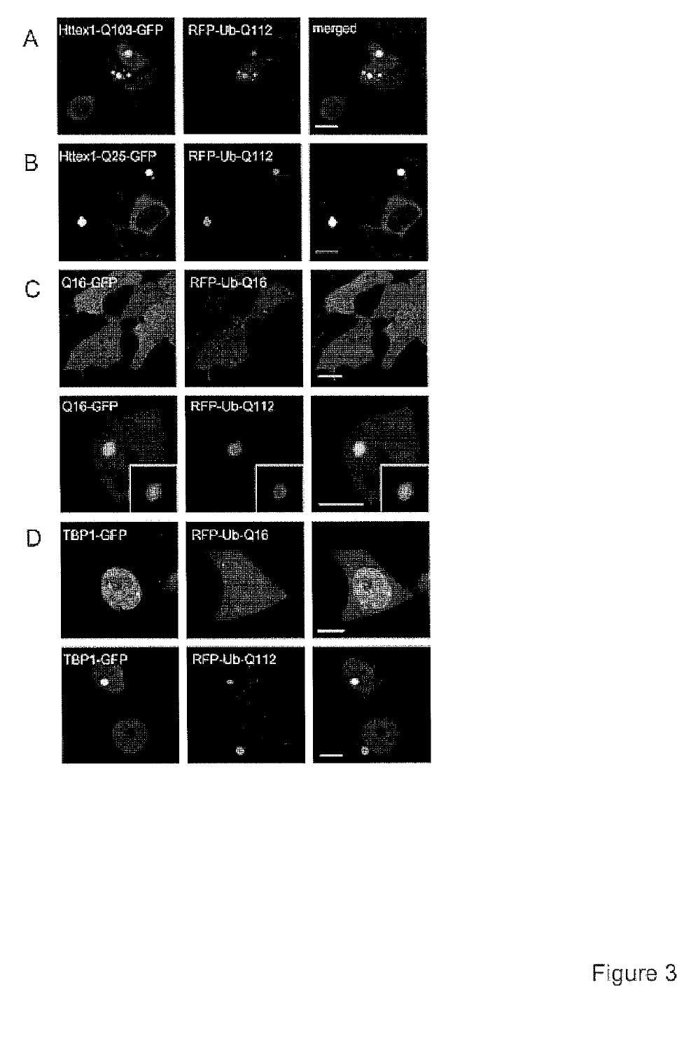

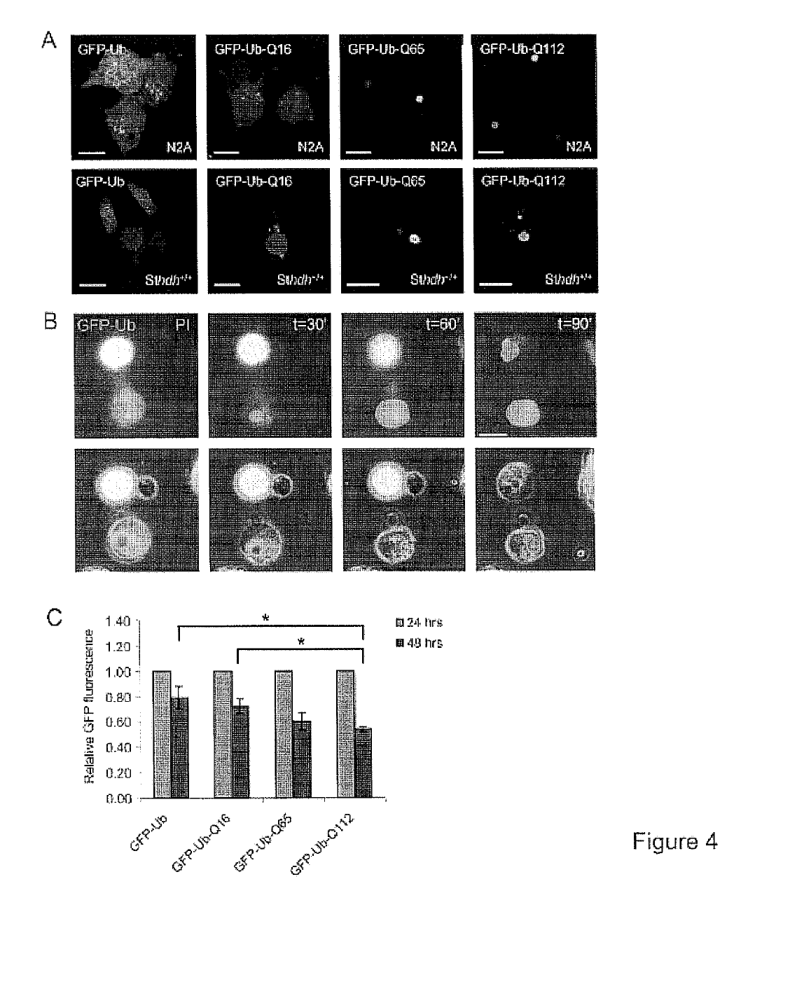

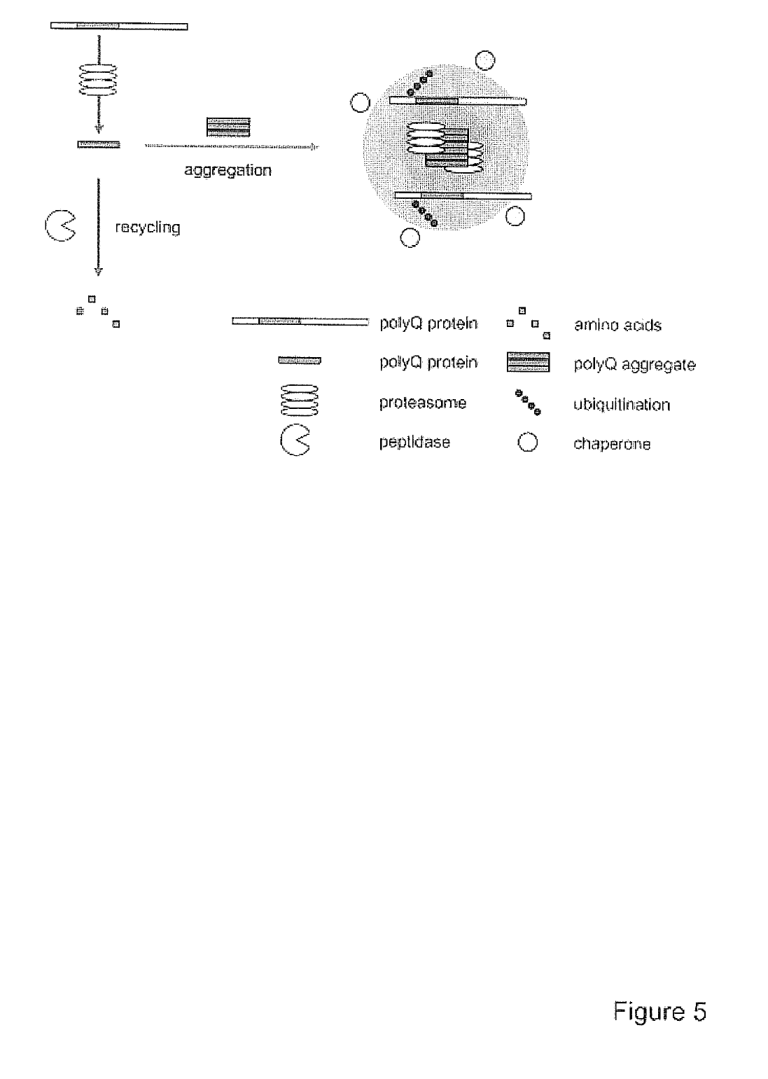

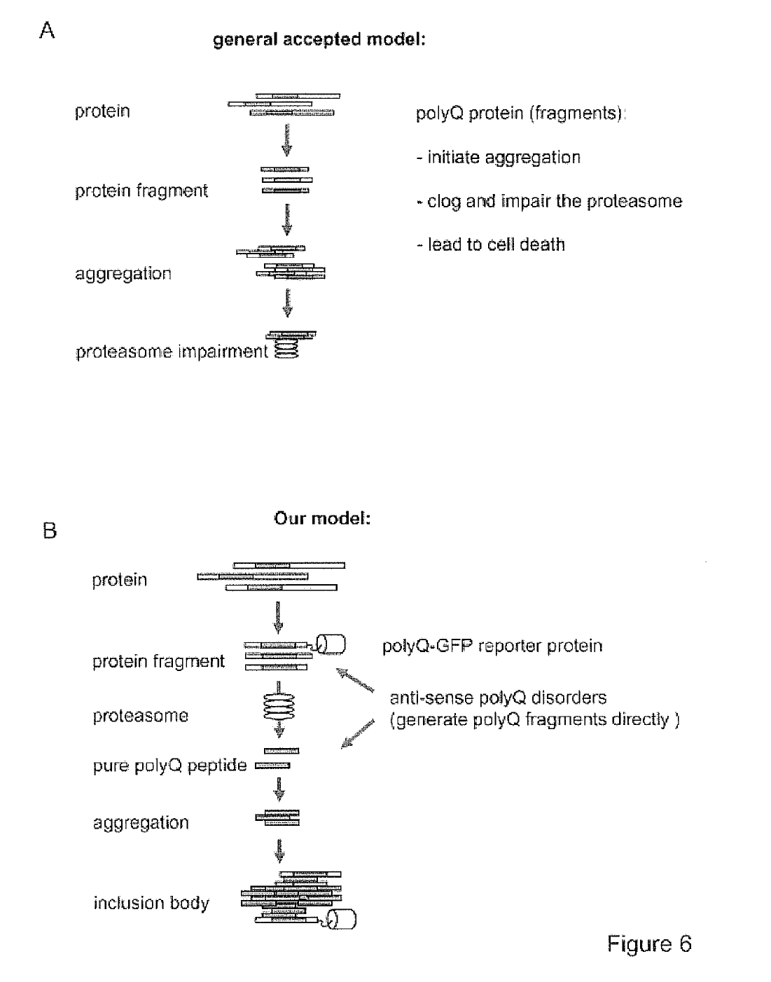

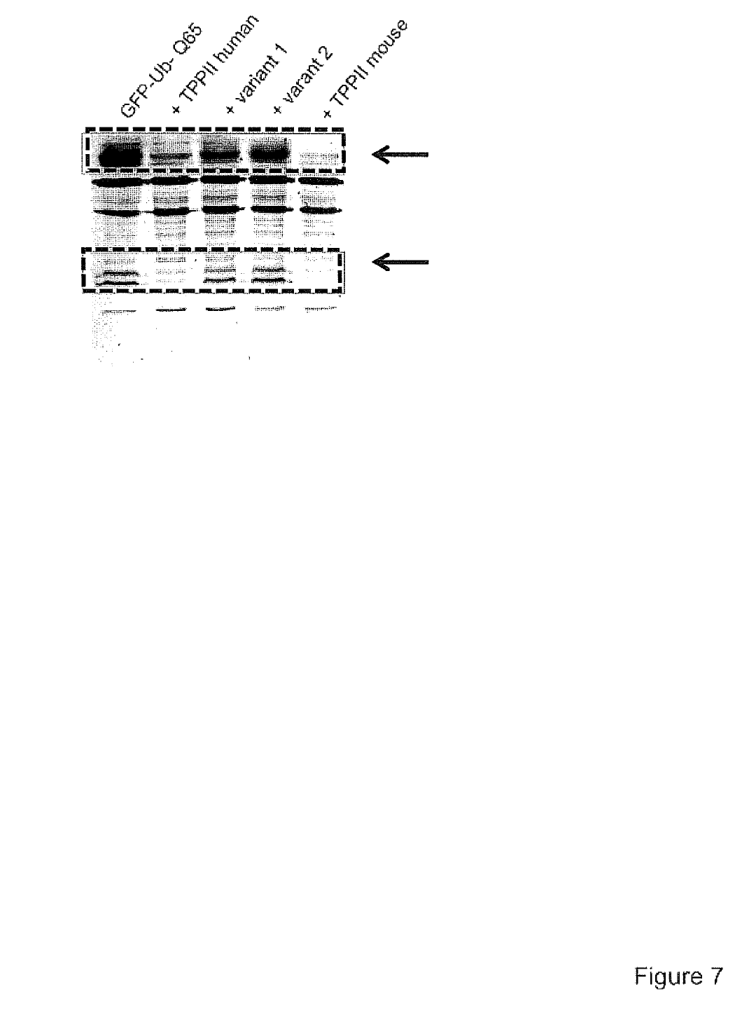

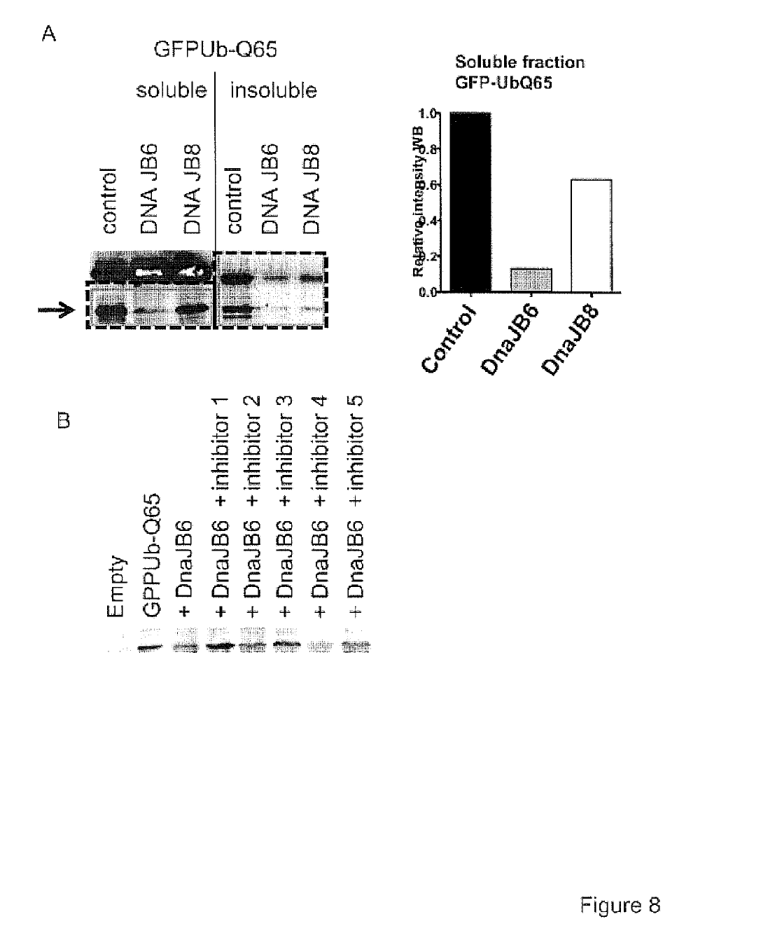

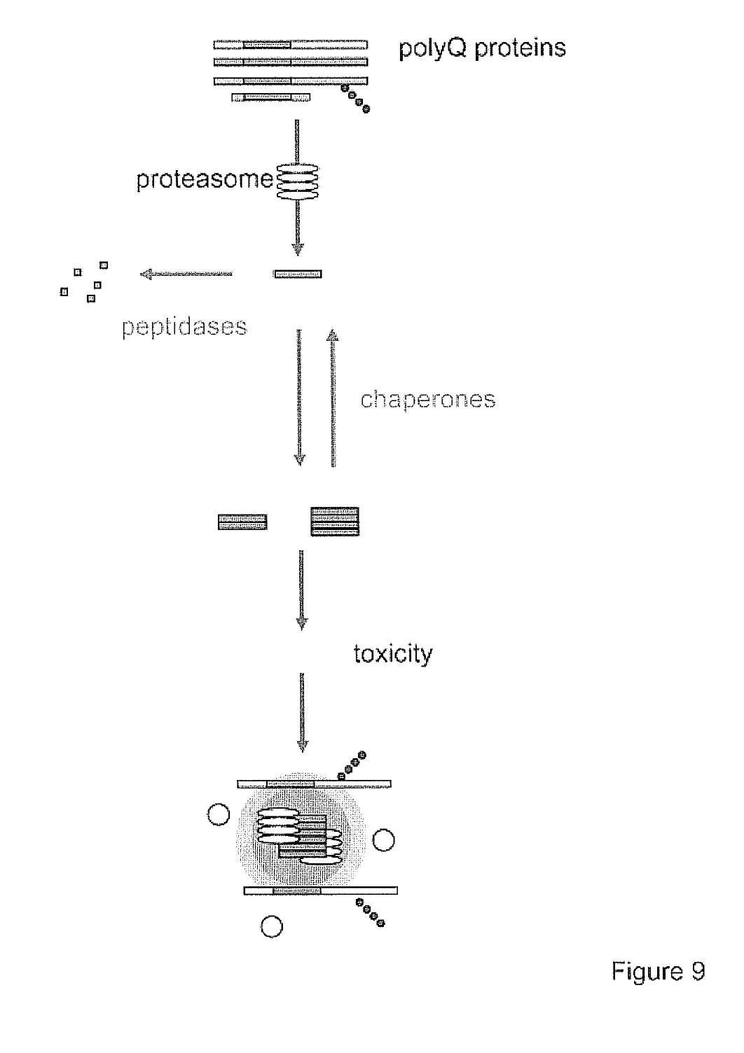

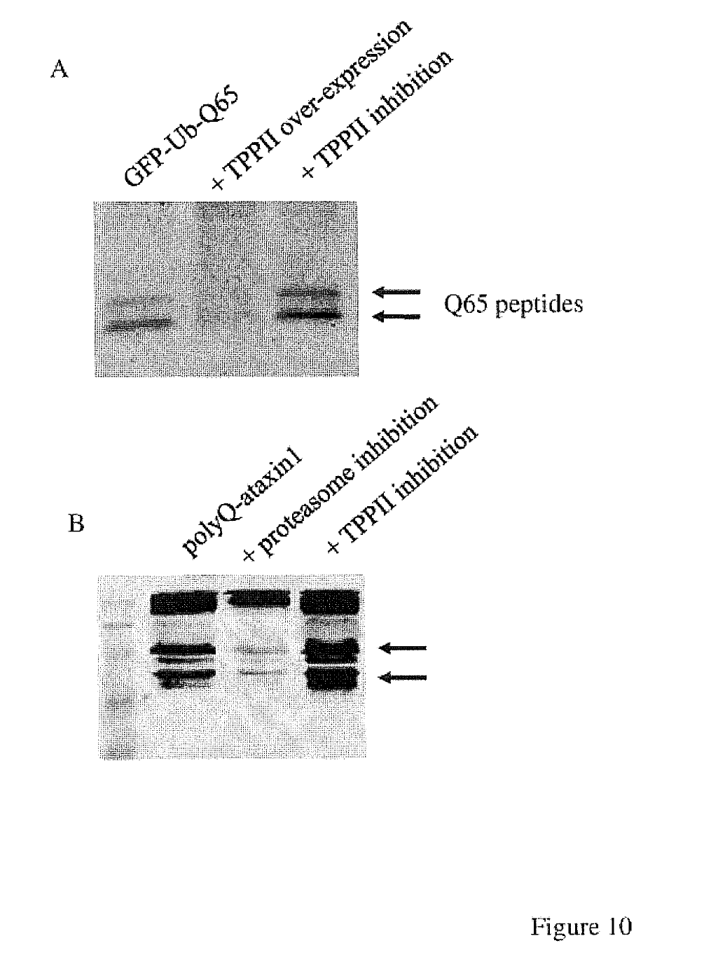

### REFERENCES

Bachmair, A., Finley, D. and Varshaysky, A. (1986). In vivo half-life of a protein is a function of its amino-terminal residue. Science 234, 179-86
Bhutani, N., Venkatraman, P. and Goldberg, A. Puromycin-sensitive aminopeptidase is the major peptidase responsible for digesting polyglutamine sequences released by proteasomes during protein degradation. The EMBO Journal 26 (2007); 1385-1396
Carra S, Seguin S J, Lambert H and Landry J. (2008). HspB8 chaperone activity toward poly(Q)-containing proteins depends on its association with Bag3, a stimulator of macroautophagy. J Biol Chem 283:1437-44.
Dantuma, N. P., Groothuis, T. A., Salomons, F. A. and Neefjes, J. (2006). A dynamic ubiquitin equilibrium couples proteasomal activity to chromatin remodeling. J Cell Biol 173, 19-26
Ko, J., Ou, S. and Patterson, P. H. (2001). New anti-huntingtin monoclonal antibodies: implications for huntingtin conformation and its binding proteins. Brain Res Bull 56, 319-29
Lindas, A-C. and Tomkinson, B. Characterization of the promoter of the gene encoding human tripeptidyl-peptidase II and identification of upstream silencer elements. Gene 393 (2007); 62-69
Nagai, Y., Tucker, T., Ren, H., Kenan, D. J., Henderson, B. S., Keene, J. D., Strittmatter, W. J. and Burke, J. R. (2000). Inhibition of polyglutamine protein aggregation and cell death by novel peptides identified by phage display screening. J Biol Chem 275, 10437-42
Qin, Z. H., Wang, Y., Sapp, E., Cuiffo, B., Wanker, E., Hayden, M. R., Kegel, K. B., Aronin, N. and DiFiglia, M. (2004). Huntingtin bodies sequester vesicle- associated proteins by a polyproline-dependent interaction. J Neurosci 24, 269-81
Reits, E. A., Benham, A. M., Plougastel, B., Neefjes, J. and Trowsdale, J. (1997). Dynamics of proteasome distribution in living cells. Embo J 16, 6087-94
Reits, E. A. and Neefjes, J. J. (2001). From fixed to FRAP: measuring protein mobility and activity in living cells. Nat Cell Biol 3, E145-7
Reits, E. A. et al. (2003). Immunity Jan;18 (1):97-108
Tomkinson, B. and Lindas, A-C. Tripeptidyl-peptidase II: a multi-purpose peptidase. The International Journal of Biochemistry & Cell Biology 37 (2005); 1933-1937
Trettel, F., Rigamonti, D., Hilditch-Maguire, P., Wheeler, V. C., Sharp, A. H., Persichetti, F., Cattaneo, E. and MacDonald, M. E. (2000). Dominant phenotypes produced by the HD mutation in STHdh(Q111) striatal cells. Hum Mol Genet 9, 2799-809
Trottier, Y., Lutz, Y., Stevanin, G., Imbert, G., Devys, D., Cancel, G., Saudou, F., Weber, C., David, G., Tora, L. et al. (1995). Polyglutamine expansion as a pathological epitope in Huntington's disease and four dominant cerebellar ataxias. Nature 378, 403-6
Verhoef, L. G., Lindsten, K., Masucci, M. G. and Dantuma, N. P. (2002). Aggregate formation inhibits proteasomal degradation of polyglutamine proteins. Hum Mol Genet 11, 2689-700
Wanker, E. E., Scherzinger, E., Heiser, V., Sittler, A., Eickhoff, H. and Lehrach, H. (1999). Membrane filter assay for detection of amyloid-like polyglutamine-containing protein aggregates. Methods Enzymol 309, 375-86
Yang, W., Dunlap, J. R., Andrews, R. B. and Wetzel, R. (2002). Aggregated polyglutamine peptides delivered to nuclei are toxic to mammalian cells. Hum Mol Genet 11, 2905-17

1. A method for treating and/or decreasing susceptibility to a protein aggregation disorder, in a subject in need thereof, comprising administering to said subject a protocol comprising pharmaceutically effective amount of
a peptidase capable of cleaving a polyQ peptide comprising at least 45 glutamine residues or a functional catalytic domain thereof, or
a nucleic acid sequence encoding said peptidase or functional catalytic domain, or
a compound capable of enhancing the expression, amount and/or peptidase activity of said peptidase.

2. The method of claim 1, wherein said disorder is selected from Huntington's disease, Dentatorubral-pallidoluysian atrophy (DRPLA), X-linked spinal and bulbar muscular atrophy (SBMA) and spinocerebellar ataxias (SCA).

3. A method for counteracting and/or preventing aggregation of a polyQ protein in a cell, comprising providing said cell with a protocol comprising
a peptidase capable of cleaving a polyQ peptide comprising at least 45 glutamine residues or a functional catalytic domain thereof, or
a nucleic acid sequence encoding said peptidase or functional catalytic domain, or
a compound capable of enhancing the expression, amount and/or peptidase activity of said peptidase.

4. The method of claim 3, wherein said protocol results in enhanced recovery of said polyQ protein.

5. A method to identify a candidate compound that counteracts protein aggregation,
the method comprising determining whether said candidate compound is capable of increasing the expression, amount and/or peptidase activity of a peptidase that cleaves a polyQ peptide comprising at least 45 glutamine residues,
wherein a compound that is capable of increasing the expression, amount and/or peptidase activity of said peptidase is identified as a compound that counteracts protein aggregation.

6. (canceled)

7. The method of any one of claim 1, 3 or 5, wherein said peptidase is a cysteine peptidase or a serine peptidase.

8. The method of any one of claim 1, 3 or 5, wherein said peptidase is TriPeptidyl Peptidase II (TPPII).

9. (canceled)

10. The method of claim 1 or 3, wherein said protocol comprises a combination of:
(a) a peptidase capable of cleaving a polyQ peptide comprising at least 45 glutamine residues or a functional catalytic domain thereof, or
a nucleic acid sequence encoding said peptidase or functional catalytic domain, or
a compound capable of enhancing the expression, amount and/or peptidase activity of said peptidase; and
(b) a DnaJ heat shock protein or a functional protein aggregation inhibiting part thereof, or
a nucleic acid sequence encoding a DnaJ heat shock protein or a functional protein aggregation inhibiting part thereof, or
a compound capable of enhancing the expression, amount and/or protein aggregation inhibiting activity of a DnaJ heat shock protein.

11. (canceled)

12. A pharmaceutical composition comprising:
(a) a peptidase capable of cleaving a polyQ peptide comprising at least 45 glutamine residues or a functional catalytic domain thereof, and/or
a nucleic acid sequence encoding said peptidase or functional catalytic domain, and/or
a compound capable of enhancing the expression, amount and/or peptidase activity of said peptidase; and
(b) a DnaJ heat shock protein or a functional protein aggregation inhibiting part thereof, or
a nucleic acid sequence encoding a DnaJ heat shock protein or a functional protein aggregation inhibiting part thereof, or
a compound capable of enhancing the expression, amount and/or protein aggregation inhibiting activity of a DnaJ heat shock protein;
and, optionally, a pharmaceutical acceptable carrier, diluent or excipient.

13. The composition of claim 12, wherein said DnaJ heat shock protein comprises DnaJB6 and/or DnaJB8.

14. The composition of claim 12, wherein said compound capable of enhancing the protein aggregation inhibiting activity of said DnaJ heat shock protein comprises histone deacetylase 4 (HDAC 4) or a functional catalytic domain thereof or a nucleic acid sequence encoding HDAC 4 or a functional catalytic domain thereof.

15. A ubiquitin-polyQ fusion construct consisting of ubiquitin and a polyQ peptide, wherein said polyQ peptide consists of glutamine residues.

16. The fusion construct of claim 15, wherein said polyQ peptide has a length of at least 10 glutamine residues.

17. The fusion construct of claim 15, wherein said ubiquitin is linked to a detectable moiety.

18. The fusion construct of claim 17 which is a GFP-Ub-polyQ construct.

19. A nucleic acid sequence encoding the fusion construct of claim 15.

20. The method of claim 5 which employs the fusion construct of claim 15.

21. The method of claim 5 which employs the nucleic acid of claim 19.

22. The method of claim 10, wherein said DnaJ heat shock protein comprises DnaJB6 and/or DnaJB8.

23. The method of claim 10, wherein said compound capable of enhancing the protein aggregation inhibiting activity of said DnaJ heat shock protein comprises histone deacetylase 4 (HDAC 4) or a functional catalytic domain thereof or a nucleic acid sequence encoding HDAC 4 or a functional catalytic domain thereof.


SEQUENCE LISTING (4 sequences)

SEQ ID NO: 1 - DNA, 29 bp, Artificial
  primer
cccgagctcagatgcagatcttcgtgaag

SEQ ID NO: 2 - DNA, 28 bp, Artificial
  primer
ctcgggccctcacccacctctgagacgg

SEQ ID NO: 3 - DNA, 27 bp, Artificial
  primer
cgcggatccatggtgagcaagggcgag

SEQ ID NO: 4 - DNA, 33 bp, Artificial
  primer
cgggaattcctgcagcccacctctgagacggag